(19) United States
(12) Patent Application Publication
(10) Pub. No.: US 2026/0286368 A1
(43) Pub. Date: Sep. 24, 2026

(54) GENETICALLY ENCODED SYSTEM FOR QUANTIFYING RUBISCO ACTIVITY IN CATALYZING CARBON FIXATION

(71) Applicant: THE SCRIPPS RESEARCH INSTITUTE, La Jolla, CA (US)

(72) Inventor: Ahmed H. Badran, San Diego, CA (US)

(21) Appl. No.: 19/167,890

(22) PCT Filed: Mar. 27, 2024
(86) PCT No.: PCT/US2024/021576

§ 371 Date: Sep. 23, 2025

Related U.S. Application Data
(60) Provisional application No. 63/492,796, filed on Mar. 29, 2023.

(51) Int. Cl. C12N 15/70; C12N 9/88; C12Q 1/527

(52) U.S. Cl. CPC C12N 15/70; C12N 9/88; C12Q 1/527; C12Y 401/01039; C12Y 402/03003

(57) ABSTRACT

The present invention provides assay systems and related methods for monitoring RuBISCO catalyzed carbon fixation in a manner that is insulated from host metabolism. The invention also provides methods for evolving specific RuBISCO enzymes to identify variants with improved activities.

### CROSS-REFERENCE TO RELATED APPLICATIONS

The subject patent application claims the benefit of priority to U.S. Provisional Patent Application No. 63/492,796 (filed Mar. 29, 2023; now pending). The full disclosure of the priority application is incorporated herein by reference in its entirety and for all purposes.

### STATEMENT OF GOVERNMENT SUPPORT

This invention was made with government support under FA9550-23-1-0116 from the Air Force Office of Scientific Research Young Investigator Program. The government has certain rights in the invention.

### BACKGROUND OF THE INVENTION

Carbon flux through the biosphere is mediated predominantly by the enzyme ribulose-1,5-bisphosphate carboxylase/oxygenase (RuBisCO), which catalyzes the capture and fixation of CO₂ in first committed step of the Calvin-Benson-Bassham (CBB) cycle. Four types of structural RubisCOs are currently distinguished, of which two types, form I (CbbLS) and form II (CbbM), are known to operate in the classical autotrophic CBB cycle. As the one of the most abundant proteins on Earth, RuBisCO-dependent fixation accounts for >90% of all globally captured CO₂, totaling ~120 Gt CO₂ per year. However, the carboxylation rate of RuBisCO hinders photosynthetic efficiency: >95% of characterized RuBisCO homologs capture only 1-10 CO₂ molecules per second (3). Beyond slow carboxylation kinetics, RuBisCO can capture O₂ instead of CO₂, leading to the formation of the byproduct 2-phosphoglycolate (2PGL) that must be detoxified in photosynthetic organisms through the energy-intensive and CO₂-emitting photorespiration pathway. Photorespiration decreases net photosynthetic efficiency by up to 50%, returning a total of ~59 Gt CO₂ to the atmosphere annually. While the promiscuous specificity of RuBisCO can be offset through CO₂-concentrating mechanisms (CCMs) to minimize carbon loss, many photosynthetic organisms devote as much as 50% of their soluble protein towards biosynthesizing RuBisCO to support sufficient metabolic flux for biomass generation.

RuBisCO is a primary target for engineering efforts to increase the efficiency of photosynthesis in crops such as wheat. Its poor biochemical characteristics have made it a leading candidate for optimization to increase photosynthetic efficiency, which would have long-term implications for agricultural crop yields and as a platform for carbon capture biotechnologies. Unfortunately, only modest gains have been realized through mining natural sequence diversity, ancestral protein reconstruction, and directed evolution. State-of-the-art intracellular strategies to improve CO₂ fixation rely on RuBisCO-dependent Escherichia coli (RDE) strains, where RuBisCO-produced 3-phosphoglycerate (3PG) complements deletion of an essential glycolytic enzyme (e.g., GapA, Pgk). However, RDE strains have impaired growth rates, a high false-positive rate, and can require extensive in vitro characterization to confirm RuBisCO hypermorph improvement. In addition, many RDE-evolved RuBisCO alleles improve solubility, folding, and/or expression rather than CO₂ capture and fixation.

Thus, there is a strong need for effective methods that can quantify RuBisCO activity and abundance in living cells while concomitantly minimizing cell viability defects and selection escape. The present invention is directed this and other unmet needs in the art.

### SUMMARY OF THE INVENTION

The invention provides assay systems and related methods for monitoring RuBISCO catalyzed carbon fixation in a manner that is insulated from host metabolism. In one aspect, the invention provides engineered E. coli cells or cell lysates that bypass the canonical glycolysis pathway and do not produce or accumulate 3-phosphoglycerate (3PG) from glycolysis. In some embodiments, these engineered cells or cell lysates contain mutations in the genome that disrupt expression of one or more functional proteins encoded by serA, eda-edd, garDPLRK, glcCDEFGB, gudPXD, allS-allD gene cassette, cdaR, ghrA, ghrB, and ytjC. In some embodiments, the cells or cell lysates additionally contain a mutation that disrupts expression of a functional protein encoded by pgk. In some embodiments, the cells or cell lysates additionally contain mutations that disrupt expression of functional proteins encoded by one or more genes selected from the group consisting of gpmA, gpmM, gapA, aceBAK, and gph. In some embodiments, the cells or cell lysates additionally contain mutations disrupt expression of functional proteins encoded by gpmA, gpmM, gapA, aceBAK, and gph. In various embodiments, mutations in the one or more mutated genes in the genome are deletions. In some embodiments, the genome of the engineered cells or cell lysates further contain a point mutation in the homohexamer interface of methylglyoxal synthase (MgsA). In some of these embodiments, the point mutation contains a V111A substitution or a S79P substitution, with the amino acid numbering being based on E. coli MgsA with Uniprot ID P0A731. In some embodiments, the engineered cell or cell lysates additionally contains in its genome one or more mutations set forth in FIG. 1. In some embodiments, the engineered cell or cell lysates additionally contains a vector expressing transcriptional repressors LacI and TetR, LuxCDE, and membrane integrity-responsive LacZ cassette.

In another aspect, the invention provides engineered E. coli cells that harbor an artificial biosynthetic pathway that produces RuBisCO substrate ribulose 1,5-bisphosphate (RuBP) with reduced toxicity. In some embodiments, the artificial biosynthetic pathway expresses a reduced level of Synechococcus elongatus phosphoribulokinase (Prk). In some embodiments, the reduced Prk level is expressed with a vector containing a low-copy replication origin, a rhamnose-dependent promoter Pᵣₕₐ, and a weakened RBS. In some embodiments, the employed Synechococcus elongatus Prk contains a R52A or a W140A mutation. In another aspect, the invention provides transcriptional biosensors for quantitatively monitoring RuBisCO abundance and enzymatic activities. The transcriptional biosensors contain (a) a phosphatase that specifically de-phosphorylate 3PG to generate glycerate, and (b) a CdaR variant that has enhanced specificity for glycerate relative to wildtype CdaR. Typically, a transcriptional biosensor of the invention is encoded by and expressed from one or two expression vectors. In some embodiments, the employed CdaR variant is CdaR^{evo1} or CdaR^{evo2}, and the employed phosphatase is TtHA0368. In some embodiments, the invention provides expression vectors that encode a transcriptional biosensor of the invention or a component thereof.

In another aspect, the invention provides engineered bacterial cells (e.g., E. coli cells) for quantitatively monitoring RuBisCO-dependent CO₂ fixation. The cells contain (a) mutations in the genome that result in bypassing canonical glycolysis pathway and no production or accumulation of 3-phosphoglycerate (3PG) from glycolysis, thereby insulating host metabolism from RuBisCO-produced products, (b) an artificial biosynthetic pathway that produces RuBisCO substrate ribulose 1,5-bisphosphate (RuBP) with reduced toxicity, and (c) a transcriptional biosensor for monitoring glycerate generated from RuBisCO-produced 3PG. In some related embodiments, the invention provides methods for quantitatively monitoring RuBisCO-dependent CO₂ fixation by a RuBisCO enzyme. The methods entail (a) expressing the RuBisCO enzyme in an engineered bacterial cell described herein, (b) culturing the cell under conditions suitable for RuBisCO catalyzed CO₂ fixation, and (c) monitoring the amount of RuBisCO-produced 3PG in the cell, thereby quantitatively monitoring RuBisCO-dependent CO2 fixation by the RuBisCO enzyme. In some of these embodiments, the amount of RuBisCO-produced 3PG is monitored by quantifying glycerate converted from RuBisCO-produced 3PG. In some other related embodiments, the invention provides methods for evolving a RuBisCO enzyme. These methods involve (a) expressing one or more variants of a reference RuBisCO enzyme in an engineered bacterial cell described herein to generate a plurality of cells, wherein each cell expresses one of the RuBisCO variants, (b) monitoring RuBisCO-dependent CO2 fixation in the plurality of cells, and (c) identifying one cell expressing a RuBisCO variant with enhanced CO2 fixation relative to a control cell expressing the reference RuBisCO enzyme, thereby evolving the RuBisCO enzyme.

A further understanding of the nature and advantages of the present invention may be realized by reference to the remaining portions of the specification and claims.

### DESCRIPTION OF THE DRAWINGS

FIG. 1. Summary of strain mutations as determined by whole genome sequencing. Mutations acquired in the genetic drift phase are shown in orange, whereas all other mutations were acquired following ALE and deletion of set #2. SNP: single nucleotide polymorphism.

FIG. 2. Comparison of native, RuBisCO-dependent, and insulated RuBisCO-biosensing E. coli metabolism. (A) Simplified representation of the glycolytic Embden-Meyerhof-Parnas (EMP) pathway, the major energy producing pathway in E. coli. EMP connects the key metabolites glucose and pyruvate through the target metabolite 3-phosphoglycerate (3PG). (B) RuBisCO-Dependent E. coli (RDE) strains delete EMP enzymes upstream of 3PG to disrupt glycolysis, ablating energy generation through glucose catabolism. EMP disruption is complemented through a RuBisCO-dependent bypass: phosphoribulokinase (Prk) generates ribulose 1,5-bisphosphate (RuBP), which RuBisCO uses to generate 3PG. Alternative metabolic bypasses through the Entner-Doudoroff pathway (ED) or RuBisCO-dependent oxygenation reactions that generate 2-phosphoglycerate (2PGL) may confound experimental interpretation. (C) RuBisCO-Biosensing E. coli (iRBE) strains similarly disrupt EMP and optimize a previously unrecognized glycolytic bypass through the methylglyoxal pathway (MG) to reestablish a connection from glucose to pyruvate. After deleting enzymes known or predicted to produce, consume, transport, and/or bind metabolites with structural or chemical similarity to 3PG and 2PGL, RuBisCO activity can be monitored using a dedicated biosensor.

FIG. 3. Analysis of engineered and evolved strain growth rates across relevant bacteriological media. Deletion set #1 is shown as orange boxes, while deletion set #2 is shown in differentially shaded purple boxes to indicate the different chemostats used for evolution. Post-evolution strain diversity was preserved while deleting set #2 (not clonal). Data reflect the mean and standard deviation of 4 biological replicates.

FIG. 4. Discovery of a 3PG-permissive phosphatase to generate glycerate in living cells. (A) Strategy for phosphatase and importer analysis. Exogenous 3PG is transported into the cytosol by an inner membrane transporter. The phosphatase cleaves 3PG to yield glycerate, which is detected by CdaR^{evo1}. (B) Spurious activation of the CdaR^{evo1} biosensor by Gph and GsPhoE in the absence of exogenous 3PG. (C) On-target CdaR^{evo1} biosensor activation after exogenous 3PG addition in a phosphatase-dependent manner. EcUhpT_{D388C} outperformed StPgtP alongside all tested phosphatases in response to 3PG. (D) CdaR^{evo1} biosensor dynamic range+/−3PG using all phosphatases and importers in response to 3PG. (E) Low-level constitutive phosphatase expression improves biosensor signal in response to 3PG. (F) Low-level constitutive phosphatase expression improves biosensor dynamic range in response to 3PG, spearheaded by YbhA (Uniprot P21829), ScTOR283w (Uniprot Q12040), and TtHA0368 (Uniprot Q5SLC5). (G) Glycerate titration analysis in S4012 cells encoding the top performing phosphatases, highlighting the undesirably high CdaR^{evo1} biosensor activation when using YbhA and ScTOR283w. (H) 3PG titration analysis in S4012 cells, showcasing phosphatase- and 3PG-dependent CdaR^{evo1} biosensor activation. Dose-dependent signal increase without phosphatase likely reflects spontaneous 3PG dephosphorylation prior to cell entry or dephosphorylation by periplasmic phosphatase(s). (I) Inactivating mutations to the putative catalytic residues of TtHA0368 ablate CdaR^{evo1} biosensor activation.

FIG. 5. T. thermus HA0368 homology and structural modeling identifies putative active site residues. (A) Alignment of T. thermus HA0368 with E. coli phosphoglycerate mutases (GpmA) and putative phosphoglycerate phosphatases (GpmB) (SEQ ID NOs:1-3). Active site residues are highlighted. (B) Alphafold-predicted (64, 65) T. thermus HA0368 structure with putative active site residues shown. The predicted structure shows a large and unobstructed pocket leading to the active site, which may influence substrate scope. (C) Crystal structure of E. coli phosphoglycerate mutase GpmA (PDB: 1E58) with active site residues shown.

FIG. 6. List of candidate genes for deletion to insulate RuBisCO products from host E. coli metabolism. Genes are grouped by the major metabolites that they affect or structural analogs thereof. Each gene was prioritized for deletion based on literature precedent and confidence in reactivity towards all four molecules. +++: high priority, ++: intermediate priority, +/−: low priority.

### DETAILED DESCRIPTION OF THE INVENTION

### I. Overview

Strategies to study and alter the biochemical properties of RuBisCO often couple CO₂ fixation to bacterial growth. However, viability-coupled strategies are not quantitative and are limited by toxicity of the RuBisCO substrate RuBP, slow kinetics of RuBisCO, and differences in RuBisCO expression. The present invention is predicated in part by the inventor to develop a tripartite approach to quantify RuBisCO-dependent CO₂ fixation in living cells. As detailed herein, the inventor developed the first genetically encoded system capable of accurately quantifying RuBisCO-dependent carbon fixation in cellulo. The tripartite platform overcame the low throughput and slow growth rate of RDE approaches by decoupling RuBisCO activity from metabolism, which was achieved by engineering E. coli to use an underexploited glycolytic bypass that eliminates the accumulation of 3PG from glycolysis. Using this metabolically insulated chassis bacterium, the inventor created an artificial biosynthetic pathway that controls the abundance of the toxic RuBisCO substrate ribulose 1,5-bisphosphate (RuBP) and converts RuBisCO-derived 3PG to glycerate. Glycerate thus produced was quantitatively monitored via an evolved transcriptional biosensor. Since RDE approaches can incentivize the evolution of increasingly soluble RuBisCOs, the inventor developed a strategy to fine-tune and quantify intracellular RuBisCO abundance, which can be used to normalize biosensor activities. The inventor further extended these resources to 43 diverse RuBisCO enzymes to find good agreement between in cellulo biosensor activities and in vitro-derived kinetic parameters. Finally, the inventor demonstrated the rapid enrichment of a functional RuBisCO enzyme from a library of inactive variants in only a few days. By showcasing how RuBisCO activity can be readily monitored and evolved in a cellular setting, the invention establishes a modern approach to quantify CO₂ fixation and provide an effective platform for the directed evolution of RuBisCO enzymes with enhanced CO₂ capture efficiencies.

In accordance with these studies, the present invention provides engineered bacterial cells that bypass canonical glycolysis pathway and does not produce or accumulate 3-phosphoglycerate (3PG) from glycolysis, expression systems encoding an artificial biosynthetic pathway that produces RuBisCO substrate ribulose 1,5-bisphosphate (RuBP) with reduced toxicity, and expressing vectors encoding a transcriptional biosensor for monitoring RuBisCO-dependent 3PG production. Also provided in the invention is an tripartite system or platform for quantitatively monitoring RuBisCO-catalyzed carbon fixation in a manner that is insulated from host metabolism, as well as methods for evolving specific RuBisCO enzymes to identify variants with improved activities.

The following description provides a more detailed guidance for practicing the various embodiments of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

Ribulose-1,5-bisphosphate carboxylase/oxygenase is commonly known by the abbreviations RuBisCo, rubisco, RuBPCase, or RuBPco. It is an enzyme (EC 4.1.1.39) involved in the first major step of carbon fixation, a process by which atmospheric carbon dioxide is converted by plants and other photosynthetic organisms to energy-rich molecules such as glucose. Rubisco can catalyze the addition of O2 to RuBP, producing 3PG, which can be used by the Calvin cycle, and 2-phosphoglycolate, which is converted to glycolate, transported out of the chloroplast, converted to glycerate over several steps in the peroxisome and mitochondria, and shipped back to the chloroplast. RubisCO is widespread and can be found in plants, algae, cyanobacteria, many autotrophic bacteria (phototrophs and chemolithotrophs), and archaea.

Substrates for RuBisCO are ribulose-1,5-bisphosphate (RuBP) and carbon dioxide (distinct from the “activating” carbon dioxide). RuBisCO also catalyses a reaction of ribulose-1,5-bisphosphate and molecular oxygen (O₂) instead of carbon dioxide (CO₂). Discriminating between the substrates CO₂ and O₂ is attributed to the differing interactions of the substrate's quadrupole moments and a high electrostatic field gradient. This gradient is established by the dimer form of the minimally active RuBisCO, which with its two components provides a combination of oppositely charged domains required for the enzyme's interaction with O₂ and CO₂. When carbon dioxide is the substrate, the product of the carboxylase reaction is an unstable six-carbon phosphorylated intermediate known as 3-keto-2-carboxyarabinitol-1,5-bisphosphate, which decays rapidly into two molecules of glycerate-3-phosphate. This product, also known as 3-phosphoglycerate (3PG), can be used to produce larger molecules such as glucose. When molecular oxygen is the substrate, the products of the oxygenase reaction are phosphoglycolate and 3-phosphoglycerate. Phosphoglycolate is recycled through a sequence of reactions called photorespiration, which involves enzymes and cytochromes located in the mitochondria and peroxisomes (this is a case of metabolite repair). In this process, two molecules of phosphoglycolate are converted to one molecule of carbon dioxide and one molecule of 3-phosphoglycerate, which can reenter the Calvin cycle.

The Calvin cycle fixes atmospheric carbon to ribulose 1,5-bisphosphate to form the organic 3-carbon intermediate 3-phosphoglycerate for the formation of sugars. Calvin cycle reactions can be organized into three basic stages: fixation, reduction, and regeneration. In the stroma, in addition to CO₂, two other chemicals are present to initiate the Calvin cycle: RuBisCO, and ribulose 1,5-bisphosphate (RuBP). RuBisCO catalyzes a reaction between CO₂ and RuBP, which forms a six-carbon compound that is immediately converted into two three-carbon compounds, 3-phosphoglycerate (3PG). This process is called carbon fixation, because CO₂ is “fixed” from its inorganic form into organic molecules. Phosphoglycerate kinase phosphorylates 3PG to 1,3-bisphosphoglycerate. 1,3-BPG is then reduced by glyceraldehyde-3-phosphate dehydrogenase to form glyceraldehyde-3-P. This type of reaction is called a reduction reaction because it involves the gain of electrons. A reduction is the gain of an electron by an atom or molecule. The molecules of ADP and NAD⁺, resulting from the reduction reaction, return to the light-dependent reactions to be re-energized. A small portion of glyceraldehyde-3-P molecules leave the Calvin cycle to contribute to the formation of the carbohydrate molecule, which is commonly glucose (C₆H₁₂O₆). Because the carbohydrate molecule has six carbon atoms, it takes six turns of the Calvin cycle to make one carbohydrate molecule (one for each carbon dioxide molecule fixed). The remaining glyceraldehyde-3-P molecules regenerate RuBP, which enables the system to prepare for the carbon-fixation step.

### III. Tripartite System for Monitoring RuBisCO-Dependent CO2 Fixation

There currently are very few effective methods for expressing functional plant Rubisco in bacterial hosts for genetic manipulation studies. This is largely due to Rubisco's requirement of complex cellular machinery for its biogenesis and metabolic maintenance including the nuclear-encoded RbcS subunits, which are typically imported into chloroplasts as unfolded proteins. Furthermore, sufficient expression and interaction with Rubisco activase are major challenges as well. In addition, examining RuBisCO activity via monitoring RuBisCO-derived 3PG and 3PG conversion into glycerate is complicated by the fact that 3PG is also a central metabolite of the host glycolytic pathway. Further, providing RuBisCO substrate RuBP by heterologously expressing Prk as required in the RDE complementation approach to monitor RuBisCO activity is faced with RuBP toxicity.

The invention provides a novel tripartite platform or system for quantitatively monitoring RuBisCO-dependent CO2 fixation. The platform employs engineered bacterial strains (e.g., E. coli cells) that utilize a bypass glycolysis pathway and insulates RuBisCO-derived 3PG from host metabolism, i.e., eliminating 3PG accumulation from host glycolysis. The engineered bacterial cells also contain an optimized RuBP biosynthetic pathway that eliminates RuBP toxicity in the cells. Further encompassed by the engineered bacterial cells is an engineered biosensor that converts RuBisCO generated 3PG into glycerate and quantifies glycerate thus generated, thereby concurrently monitoring RuBisCO abundance and catalysis.

Functionality of the tripartite system is demonstrated herein by intracellular quantification of RuBisCO activity and enzyme concentration, which enables rapid validation of in vitro-derived kinetic parameters and functional enzyme enrichment. As detailed in the Examples, the inventors examined unrooted phylogenetic tree of 43 previously characterized Form II and II/III RuBisCO variants. Using an in vitro enzyme coupled assay, CO₂ capture and subsequent 3PG generation (in the presence of an appropriate CO₂ concentration, e.g., at 10% CO₂) by the RuBisCO variants were analyzed. As exemplified herein, RuBisCO abundance in the employed E. coli strain can be measured and normalized by monitoring a fluorescent signal due to a sfGFP11 tag at the C-terminus of the enzyme. Activation of a CdaR variant based biosensor (e.g., CdaR^{evo1} or CdaR^{evo2}) using the 43 RuBisCO variants can be compared without or after normalization for RuBisCO abundance. As described in the Examples, the results indicate that agreement between the CdaR biosensor activation and in vitro derived kinetics is improved to provide a more accurate quantification of intracellular bioactivity.

As demonstrated herein, the assay platform developed by the inventor overcomes prior limitations by streamlining RuBisCO analyses, and finds utility in studies to explore and improve RuBisCO-dependent CO₂ fixation. In some embodiments, the assay system can be used in methods for evolving functional RuBisCO enzymes. As exemplification, an NNK degenerate library randomizing all CbbM essential residues K191, D193 and E194 can be created by PCR using primers synthesized to be degenerate at those positions. The PCR can be performed using the inactive template CbbM (K191M) to minimize contamination in downstream steps. The fragment library can then be USER-assembled and transformed into an engineered E. coli cell described herein (e.g., S5643) that harbors the other components of the tripartite system. These include optimized phosphoribulokinase (variant PrkA(R52A)) to generate RuBP, phosphatase TtHA0368 to convert 3PG to glycerate, and CdaR^{evo1} for sensing glycerate. After culturing and incubation in the presence of CO₂, the cells are analyzed via fluorescence activated cell sorting (FACS). Upon confirmation of positive signals, RuBisCO plasmids can be isolated from functional cultures and subjected to further examination (e.g., sequencing analysis).

In addition to a living cell based setting, the tripartite system of the invention for monitoring RuBisCO-dependent CO2 fixation can also be in a cell-free setting. In these embodiments, cell lysates can be prepared from the engineered bacterial cells expressing the relevant biological pathways and biosensors described herein. Activities of a target RuBisCO or variants thereof that need to be examined or evolved can then be assessed and monitored in the cell lysates in vitro.

### IV. Engineered Bacterial Strains with Metabolic Insulation of RuBisCO-Derived Products

One aspect of the invention is directed to engineered bacterial cells that bypass canonical glycolysis pathway and do not produce or accumulate 3-phosphoglycerate (3PG) from glycolysis. By insulating RuBisCO-derived products from host-encoded metabolism (e.g., accumulation of 3PG from glycolysis in the host), such engineered bacterial strains (e.g., E. coli) are essential to constructing the tripartite system of the invention for monitoring RuBisCO-dependent CO2 fixation and for evolving RuBisCO. By decoupling RuBisCO activity from host metabolism, the engineered bacterial strains enable the development of dedicated and quantitative biosensors for CO₂ fixation.

In some preferred embodiments, the engineered bacterial strains are E. coli strains. These engineered E. coli strains can be produced in accordance with the methods described herein. As exemplifications, RuBisCO-insulated E. coli strains were generated via metabolic engineering and adaptive laboratory evolution. Examples of such E. coli strains include, e.g., strains S4003, S4006, S4221, S4009, and S4012. As detailed in the Examples, these strains are able to insulate RuBisCO-derived 3PG and 2PGL from host metabolism.

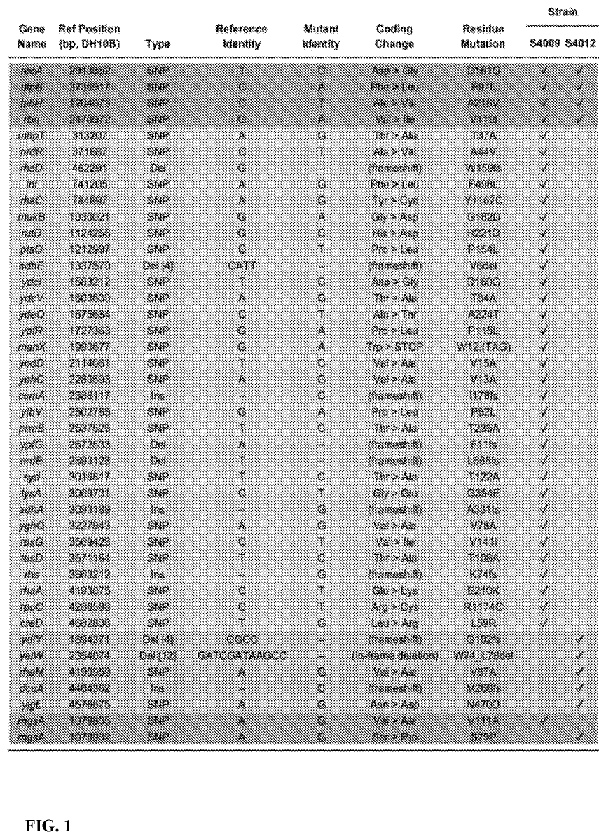

Detailed genotypes of several specific engineered E. coli strains are described herein (Table 2), including the specific mutations in the genome. In various embodiments, the engineered E. coli cells contain mutations that disrupt expression of one or more functional proteins encoded by serA, eda-edd, garDPLRK, glcCDEFGB, gudPXD, allS-allD gene cassette, cdaR, ghrA, ghrB, and ytjC. In some embodiments, the cells additionally contain a mutation that disrupts expression of a functional protein encoded by pgk. In some embodiments, the cells additionally contain mutations that disrupt expression of functional proteins encoded by one or more genes selected from the group consisting of gpmA, gpmM, gapA, aceBAK, and gph. In some preferred embodiments, the mutations in the genome of the cells are deletions. In some embodiments, the genomes of the cells additionally contain a point mutation in the homohexamer interface of methylglyoxal synthase (MgsA). In some of these embodiments, the point mutation in MgsA is a V111A substitution or a S79P substitution, wherein the amino acid numbering is based on E. coli MgsA protein with Uniprot ID P0A731 (NCBI Reference Sequence NP_415483.2). Some engineered E. coli cells of the invention contain in their genomes the mutations set forth in FIG. 1. Some of these cells can also harbor an exogenously introduced vector that expresses transcriptional repressors LacI and TetR, LuxCDE, and membrane integrity-responsive LacZ cassette.

### V. Artificial Biosynthetic Pathway to Reduce RuBP Toxicity

The RuBisCO substrate RuBP is not natively biosynthesized by E. coli. Heterologously expressed Prk is required to phosphorylate ribulose-5-phosphate (Ru5P), a pentose phosphate pathway intermediate, to generate RuBP in E. coli. However, Prk activity is toxic to E. coli. The avoid or minimize RuBP toxicity associated with conventional RuBisCO monitoring systems, the tripartite system of the invention utilizes an artificial biosynthetic pathway that produces RuBisCO substrate ribulose 1,5-bisphosphate (RuBP) with reduced toxicity. In some preferred embodiments, the artificial biosynthetic pathway expresses a reduced level of the phosphoribulokinase Prk, which an essential photosynthetic enzyme that catalyzes the ATP-dependent phosphorylation of ribulose 5-phosphate (RuP) into ribulose 1,5-bisphosphate (RuBP). Typically, this artificial biosynthetic pathway is encoded by an appropriate expression vector. In some of the embodiments, the expressed Prk enzyme is Synechococcus elongatus Prk (Uniprot Q9LBV7), and the reduced Prk level is expressed with a vector containing low-copy replication origin, a rhamnose-dependent promoter Pᵣₕₐ, and a weakened RBS. As demonstrated herein, this artificial biosynthetic pathway can be readily incorporated into the engineered bacterial strain with metabolic insulation described above.

Metabolically insulated E. coli cells expressing the biosynthetic pathway can be generated by the specific protocols described herein. As detailed in the Examples, the tripartite system of the invention eliminates toxicity and enables biosensor-dependent RuBisCO monitoring in living cells by genetic and biochemical Prk tuning. The specific Prk expression plasmid exemplified herein encodes the low copy number RK2 origin, weakened RBSs, and tighter transcriptional control through the rhamnose promoter. Additionally, by introducing targeted mutations to the ATP or Ru5P binding sites, activity of S. elongatus Prk can be kinetically tuned to eliminate toxicity while still providing sufficient RuBP for RuBisCO. Specific residues for Prk mutagenesis in the ATP and Ru5P binding sites to eliminate toxicity are detailed herein. In some embodiments, the employed Prk active site mutant is Prk R52A mutant (Ru5P binding site) or Prk W140A mutant (ATP binding site). In some other embodiments, Prk enzymes from other organisms containing mutations homologous to the mutations in the exemplified S. elongatus Prk can be used.

### VI. Transcriptional Biosensors for Monitoring RuBisCO Activities

The invention provides novel transcriptional biosensors that can be employed to monitor RuBisCO catalyzed 3PG production, thereby quantitatively monitoring RuBisCO abundance and enzymatic activities. While no known biosensors respond to 3PG, the transcriptional biosensors of the invention utilize transcriptional activator CdaR, which responds to the dephosphorylated counterpart of 3PG, glycerate. Because there are no known phosphatases that act on 3PG in vivo, the CdaR-dependent transcriptional biosensors also require a phosphatase to convert 3PG into glycerate. Thus, the transcriptional biosensors of the invention typically contain (a) a phosphatase that specifically de-phosphorylates 3PG to generate glycerate, and (b) a CdaR variant that has enhanced specificity for glycerate relative to a wildtype CdaR or another known CdaR mutant. In some embodiments, the employed CdaR variant is CdaR^{evo1} or CdaR^{evo2}, which are E. coli CdaR variant generated by directed evolution as described herein. In some embodiments, the phosphatase used in the transcriptional biosensor is selected from T. thermophilus phosphatase HA0368 (TtHA0368), E. coli phosphatases Gph and YbhA, and S. cerevisiae phosphatases Pho13 and YOR283w. As exemplified herein, these enzymes can all activate CdaR^{evo1} in a 3PG-dependent manner. Typically, the transcriptional biosensor is encoded by and expressed from appropriate expression vectors. In various embodiments, the phosphatase and the CdaR variant can be expressed from two separate vectors or expressed together from the same vector, as exemplified herein.

As described herein, the transcriptional biosensors can be incorporated into the engineered bacterial cells as described above to enable quantitative monitoring of RuBisCO-dependent CO₂ in living cells. Accordingly, some related embodiments of the invention are directed to engineered cells that express a transcriptional biosensor described herein. As exemplifications, a glycerate-specific biosensor compatible with the metabolically insulated strains described herein was generated. Phosphatase TtHA0368 was obtained by monitoring on-target CdaR^{evo1} biosensor activation in S4012 cells, after exogenous 3PG addition in the presence of one of a number of phosphatases. In the optimized glycerate-responsive biosensor, CdaR is constitutively expressed and requires glycerate (GLY) to activate P_{garP} transcription, resulting in a luminescent signal through LuxAB. As detailed in the Examples, the single plasmid glycerate-responsive biosensor improves dynamic range in S4221 cells. For positive selection in the presence of glycerate, barnase is inhibited by the constitutively expressed antitoxin barstar, and glycerate-dependent activation produces a fluorescent signal (mCherry) and chloramphenicol resistance (Cat). CdaR variants with enhanced specificity for glycerate can be obtained via CdaR directed evolution as detailed herein. As described in the Examples, directed evolution of CdaR in DRM yielded variants with greatest biosensor activation. The evolved CdaR variants can exhibit two phenotypes, CdaR^{evo1} and CdaR^{evo2}. They contain both coding and non-coding mutations in the CdaR diacid binding domain. Specifically, CdaR^{evo1} contains I21F, G50C, C109R and M122K coding mutations (and A11A and I141I non-coding mutations), and CdaR^{evo2} contains R20C, C109G and G118H coding mutations (and L84L non-coding mutation). As demonstrated herein, the CdaR^{evo1} variant can restore glycerate-dependent biosensor activity in the metabolically insulated strains S4009 and S4012.

As described in the Examples, the exemplified RuBisCO biosensor was able to converts Ru5P to RuBP, which is then combined with carbonic anhydrase (CA)-derived CO₂ by a functional RuBisCO to yield two molecules of 3PG. Phosphatase TtHA0368 dephosphorylates 3PG to glycerate, resulting in CdaR^{evo1} biosensor activation. It was further shown that mutation of single or multiple active site residues of the RuBisCO (R. rubrum CbbM) enzyme to alanine ablates biosensor activity, confirming that RuBisCO-dependent 3PG generation is necessary to achieve robust biosensor activation.

### EXAMPLES

The following exemplified embodiments are provided to further illustrate the invention but not to limit its scope. Other variants of the invention will be readily apparent to one of ordinary skill in the art and are encompassed by the appended claims.

### Example 1 Metabolic Reprogramming Insulates RuBisCO Products

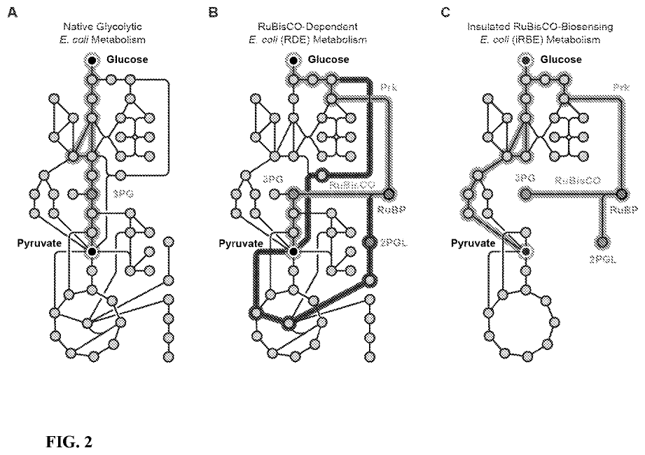

RuBisCO captures CO₂ by catalyzing the formation of a new carbon-carbon bond with RuBP, where the product spontaneously breaks down to yield two molecules of 3-phosphoglycerate (3PG). Since 3PG is a central metabolite in the E. coli Embden-Meyerhof-Parnas (EMP) pathway (FIG. 2, A), RDE approaches use this relationship to evolve RuBisCO: heterologously expressed phosphoribulokinase (Prk) generates RuBP, which RuBisCO carboxylates and cleaves to generate two molecules of 3PG in an EMP-deficient E. coli strain (FIG. 2, B) (13). Rather than relying on this complementation strategy, we sought to insulate RuBisCO-derived products from host-encoded metabolism (FIG. 2, C) to enable the development of dedicated and quantitative biosensors for CO₂ fixation.

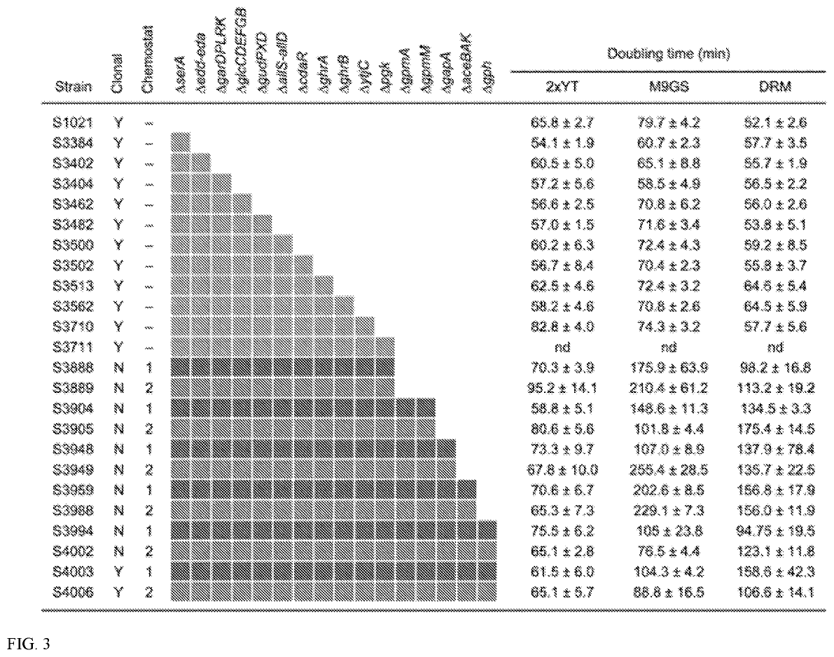

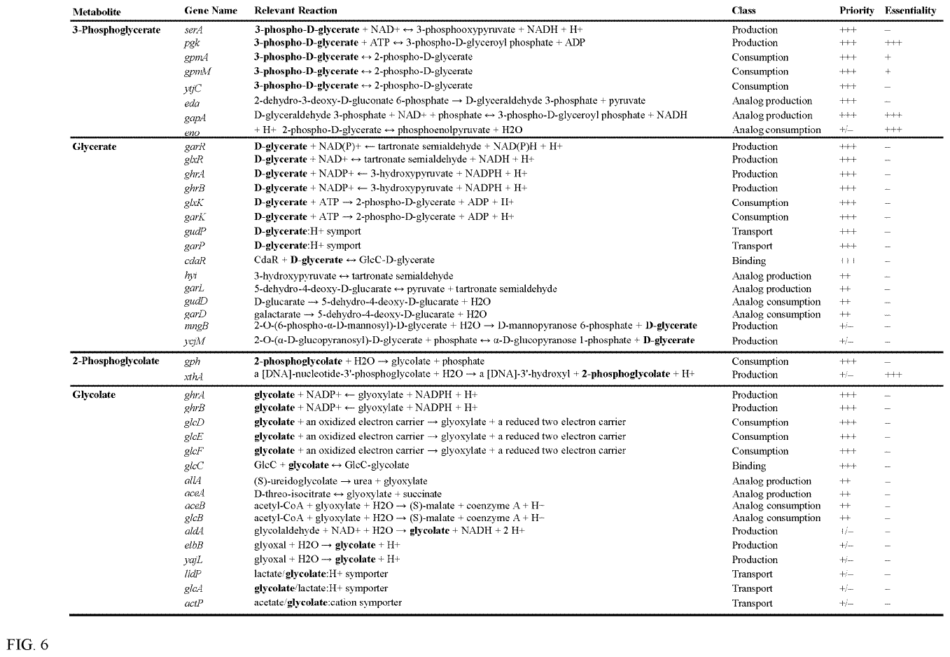

We first consulted the STRING (19) and EcoCyc (20) databases to nominate proteins known or predicted to produce, consume, transport, and/or bind metabolites with structural or chemical similarity to 3PG or the product of oxygen fixation, 2-phosphoglycolate (2PGL). This analysis yielded 39 candidate essential and non-essential genes for deletion (FIG. 6) (21). We first pursued deletions of all high priority non-essential genes in E. coli strain S1021 (22) using a λ-red recombineering strategy that permits positive-negative selection for scarless genome editing (Table 1) (23). This strategy gave rise to E. coli strain S3710, which grew comparably to S1021 (FIG. 3). Next, we explored deletions of the relevant (and essential) EMP genes: gapA, pgk, gpmA, and gpmM. E. coli mutants lacking these genes can have reduced fitness and are often sensitive to glucose due to glycolytic metabolite buildup (24), but remain viable using minimal media supplemented with glycerol and succinate (e.g., M9GS) (25). Using M9GS and S3710 cells, we deleted pgk to afford the E. coli strain S3711 and confirmed its sensitivity to glucose.

Further genome editing of S3711 cells was not possible, perhaps reflecting unknown metabolic dependencies in M9GS. However, no alternative medium formulation improved S3711 fitness beyond M9GS. We therefore evolved S3711 cells through adaptive laboratory evolution (ALE) to restore its fitness and enable additional genetic deletions, and chose to use Davis Rich Medium (DRM) (22) since it has been previously used to effectively grow E. coli in chemostat settings with optimal resource economy. We first cultured S3711 cells in DRM^{A} (phosphate-buffered amino acids and nucleobases) with glycerol and succinate to incentivize genetic drift, which we envisioned would enable the discovery of mutations with improved fitness in glucose-rich medium downstream. In order to minimize the stringency of this selection, we titrated complete DRM (contains 25 mM glucose) (22) at an initial concentration of 0.001% to maintain the glucose concentration below the 1 μM toxicity threshold (26). We progressively doubled the DRM concentration to 100% over 22 days, and then increased the flow rate to 2 vol h⁻¹ over 33 days to ensure that evolved cells could readily grow in this medium at an appreciable rate. Following daily sampling throughout the 62-day ALE campaign, we quantified cell titer on DRM^{A} with glycerol/succinate, DRM, and the standard laboratory medium 2×YT. Interestingly, both chemostat populations showed a marked fitness increase in DRM and 2×YT at 13-14 days.

Chemostat populations at 62 days were used for subsequent genome modification to preserve allelic diversity. We deleted all remaining candidate glycolytic enzymes as well as gph and aceBAK (to eliminate glycolate and glyoxylate production, respectively) from the E. coli genome, corresponding to 31 genes across 46,640 base pairs in total. We noted that these deletion efforts culminated in a phenotypically distinct strain from each chemostat population: S4003 and S4006. Whole genome sequencing revealed that each isolate independently converged on a unique mutation to methylglyoxal synthase (MgsA), the rate limiting enzyme in the methylglyoxal bypass (FIG. 1). Whereas methylglyoxal is a toxic electrophile that can irreversibly modify proteins (27), it is thought to affect the transition the cell's metabolic profile between conditions of resource abundance and starvation (28). Both mutations in mgsA map to the homohexamer interface, which regulates allostery and cooperativity during catalysis (29). Since both strains can robustly grow in medium containing glucose but did not acquire mutations predicted to limit glucose uptake (FIG. 1), we speculate that the methylglyoxal pathway now supports an effective glycolytic bypass, a hypothesis corroborated by a recent finding that MgsA overexpression permitted growth on glycerol in an EMP-deficient E. coli (30). Finally, we completed our strain development efforts by engineering an F′ plasmid, pOX38^{int} (based on pOX38::Tc) (31), that expresses key proteins that would streamline our reporter assays: the transcriptional repressors LacI and TetR for tunable promoter control, LuxCDE to generate the LuxAB substrate decanal in situ, and the membrane integrity-responsive LacZ cassette (32). We mated the key strains S3711, S4003, and S4006 with the pOX38^{int} donor strain S3926 to create S4221, S4009, and S4012, respectively (Table 2). These mated strains were used for all subsequent assays.

### Example 2 Development of a Glycerate-Responsive Biosensor

Whereas RDE systems couple CO₂ fixation to cell fitness (12-15), we aimed to monitor RuBisCO product formation using a biosensor strategy to streamline future characterization of CO₂ capture efficiency. While no known biosensors respond to 3PG, the transcriptional activator CdaR responds to its dephosphorylated counterpart glycerate (33, 34). We reasoned that it may be combined with a phosphatase to quantify 3PG production by RuBisCO. We tested CdaR-responsive promoters (33, 34) in S4221 cells alongside CdaR overexpression from a second plasmid, finding that P_{garP} responded most sensitively to glycerate. While this dynamic range could be improved by condensing the genetic circuit to a single plasmid in S4221 cells, P_{garP} was constitutively activated in the evolved S4009 and S4012 strains following ALE metabolic optimization. We hypothesized that CdaR was activated by an unknown off-target metabolite following ALE, and thus developed a dual positive-negative selection scheme to evolve CdaR variants with greater specificity for glycerate. After one round of error-prone PCR and selection, we uncovered CdaR mutants with glycerate-dependent reporter fluorescence, where isolates evolved in DRM outperformed those from 2×YT. Sequencing of the best performing clones from DRM and 2×YT revealed convergence on mutations predicted to affect a putative interfacial ligand-binding pocket. The DRM-derived consensus variant CdaR^{evo1} reestablished a large glycerate-dependent dynamic range in S4012 cells across multiple reporter contexts (35-37), nominating this mScarlet-I biosensor-strain combination as the leading chassis for further biosensor development.

### Example 3 Broad-Scope Phosphatases Convert 3PG to Glycerate

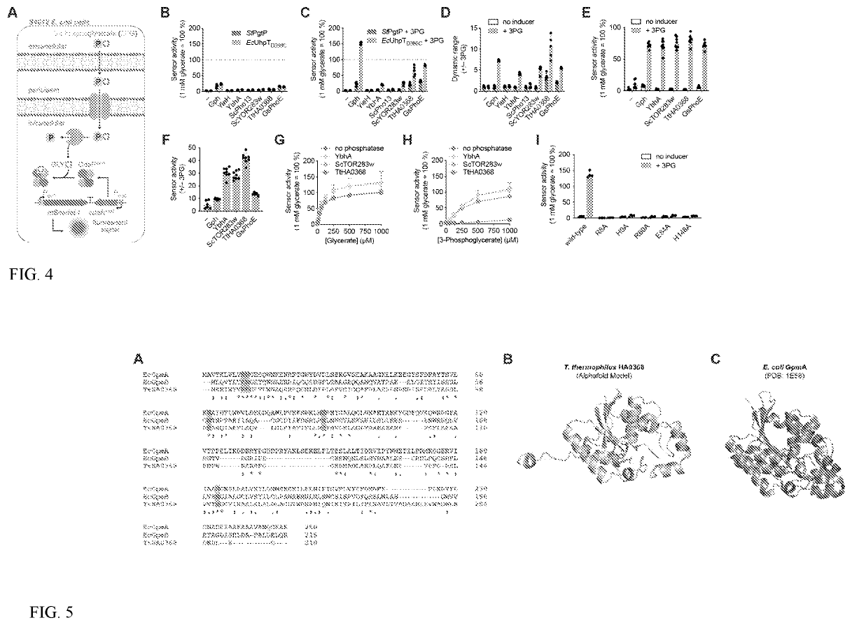

CdaR^{evo1}-dependent detection of RuBisCO activity requires dephosphorylation of 3PG to yield glycerate, yet no phosphatases that act on 3PG in vivo have been reported to date. Since exogenous 3PG cannot traverse cell membranes (38), we expressed S. typhimurium PgtP (StPgtP) (38) and E. coli UhpT_{D388C} (EcUhpT_{D388C}) (39) to import exogenous 3PG into cells (FIG. 4, A). To establish our approach we tested inducible phosphatases that are known to dephosphorylate 3PG in vitro: E. coli Gph, YieH and YbhA (40); Saccharomyces cerevisiae ScPho13 (41) and ScYOR283w (42); Thermus thermophilus TtHA0368 (43); Geobacillus stearothermophilus GsPhoE (43). Whereas Gph and GsPhoE non-specifically activated CdaR^{evo1} (FIG. 4, B), the remaining enzymes Gph, YbhA, ScYOR283w, TtHA0368, and GsPhoE all activated CdaR^{evo1} in a 3PG-dependent manner (FIG. 4, C, D), with EcUhpT_{D388C} outperforming StPgtP. Constitutive expression of these five phosphatases prior to 3PG incubation further amplified CdaR^{evo1}-dependent dynamic range (FIG. 4, E, F). To nominate the most effective phosphatase, we titrated the top performing enzymes YbhA, ScTOR283w, and TtHA0368 with glycerate and 3PG. YbhA and ScTOR283w activated CdaR^{evo1} to a greater extent than the no-phosphatase control across all glycerate concentrations, suggesting an unintended false-positive impact on biosensor performance. This effect was consistent in the 3PG titration, whereas TtHA0368 showed the predicted response in both settings (FIG. 4, G, H). We confirmed the on-target activity of TtHA0368 by nominating putative catalytic residues through homology and structural modeling (FIG. 5, A-C) and show that mutating active site residues abolishes phosphatase activity and CdaR^{evo1}-dependent signal generation. These results establish that CdaR^{evo1} and TtHA0368 can be used together to quantify intracellular 3PG concentrations in metabolically-insulated E. coli strains.

### Example 4 Kinetic Tuning Alleviates Prk Toxicity

The RuBisCO substrate RuBP is not natively biosynthesized by E. coli, which requires the expression of phosphoribulokinase (Prk) and RuBisCO to complete the Calvin Benson Bassham cycle. Heterologously expressed Prk can phosphorylate ribulose-5-phosphate (R5P), a pentose phosphate pathway intermediate, to generate RuBP in E. coli (13). However, Prk activity is toxic to E. coli and results in frequent selection escape in RDE strains (12-15). We hypothesized that this toxicity is exacerbated by efficient, high-level expression Prk constructs used in prior reports, which may be further confounded by stochastic induction of the P_{BAD} promoter in cells lacking constitutive arabinose importer expression (44). We addressed these issues by reducing Synechococcus elongatus Prk expression in E. coli using the low-copy RK2 origin (45), the tightly controlled rhamnose-dependent promoter Pᵣₕₐ (46), and a weakened RBS series (47), but found that all designs still induced toxicity in S4012 cells. We hypothesized that mutations to the ATP or R5P binding sites (48) could kinetically tune Prk activity to eliminate toxicity while still providing sufficient RuBP for RuBisCO. To concurrently investigate both points, we combined the Rhodospirillum rubrum RuBisCO (CbbM), R. rubrum carbonic anhydrase (CA) (49), TtHA0368, and CdaR^{evo1} biosensor with Prk mutants (48) driven by a weak promoter, then monitored biosensor signal generation under ambient (0.04%) and high CO₂ (5%) conditions to confirm full pathway activity. The CO₂ concentration-dependent biosensor signal from this 5-plasmid genetic circuit confirmed full pathway activity and showed that Prk mutants R52A and W140A were well tolerated. These results further established a narrow window of 15-45% wild-type Prk activity as necessary to provide sufficient RuBP for RuBisCO-dependent CO₂ fixation without affecting cell viability. To confirm that the observed signal was dependent on a functional RuBisCO, we inspected the CbbM structure and nominated conservative mutations to key residues in the CbbM active site, where all mutants and combinations thereof ablated activity. Surprisingly, CA activity (which is often included in RDE strains to increase intracellular C02 concentrations) (49) was not necessary as active site mutations predicted to eliminate its catalytic activity did not influence biosensor readout. CA was therefore removed from downstream assays. Taken together, these results establish the first biosensor capable of monitoring RuBisCO-dependent CO₂ fixation in a living cell.

### Example 5 Systematic Medium, Strain, and Circuit Optimization Improve Performance

Having established our RuBisCO-dependent biosensor, we next sought to optimize our system for future analyses. We explored the medium dependence of our biosensor-strain combination, in part due to the outcomes of our CdaR evolution campaign. We found that S4012 cells carrying all 5 plasmids grew to different densities across common microbial media, with unique impacts on biosensor activity and dynamic range. By comparing outgrowth conditions that maximized cell density with assay conditions that retained biosensor dynamic range, we identified that DRM supplemented with peptone, tryptone, or yeast extract greatly improved cell density, but also inadvertently activated the biosensor at high concentrations. Using an unbiased medium optimization approach, we further uncovered that the phosphoenolpyruvate (PEP) transition-state analog oxalate (50) was a promising additive that improved S4012 cell growth. These optimizations together gave rise to enhanced DRM (eDRM), a DRM-based medium that is supplemented with peptone, yeast extract, and oxalate at specific concentrations that maximize cell density without affecting biosensor accuracy or dynamic range (see Example 7).

We next sought to validate R. rubrum CbbM activity on eDRM agar plates. Curiously, we noted spurious activation of the mScarlet-I signal in cells encoding the catalytically inactive CbbM mutant K191M when plated near cells expressing the functional wild-type allele. This finding suggested that glycerate may passively diffuse or be actively transported from a functional RuBisCO-encoding strain to a non-functional counterpart, which would result in a high false-positive discovery rate in future directed evolution studies. To establish the responsible transporter(s), we generated glycerate donor and acceptor strains that could be orthogonally monitored using mScarlet-I and mEmerald biosensors, respectively. Next, we deleted transporters from the S4012 genome based on their reported ability to transport ligands with chemical similarity to glycerate (Table 3) and tested them using an agar-based diffusion assay. This experiment confirmed that the lactate/glycolate permease LldP was responsible for glycerate entry in S4012 cells. The resulting LldP-deleted strain S5643 (S4012 ΔlldP::kan) was used for all subsequent analyses. Finally, we condensed the prk(R52A) and cdaR^{evo1} genes into a low-copy SC101 plasmid and cultured all strains henceforth in CO₂-rich conditions without a CA expression plasmid, resulting in a three-plasmid biosensor system with reduced cellular burden.

### Example 6 Quantitative RuBisCO Benchmarking and Enrichment

We extended this second-generation biosensor to 43 Form II and II/III RuBisCO homologs (51), which we hypothesized could be used to confirm in vitro-derived enzyme kinetics in living cells for the first time. Since each homolog may be differentially expressed E. coli (8, 13), we leveraged a split sfGFP tagging strategy to quantify RuBisCO concentration during analysis (52). Whereas most RuBisCO homologs activated the glycerate biosensor, mScarlet-I signal moderately correlated with in vitro activity (r=0.2795; p=0.0731). Normalization of biosensor readout by RuBisCO abundance (via sfGFP reassembly) resulted in an improved correlation with previously reported kinetic rate constants for CO₂ capture (r=0.4037, p=0.0080). Based on these findings, we envisioned that this biosensor may be used to evolve RuBisCO enzymes with altered properties. To demonstrate this, we used a site-saturation approach to create degenerate NNK libraries at the catalytic residues K191, D193, and E194 in the R. rubrum CbbM RuBisCO active site (53) and attempted to enrich for the functional alleles via FACS. Prior biochemical analysis suggests that all three positions must converge on the wild-type amino acids to be functional, such that only a single variant in the >32,000-member library would be active. Following FACS and confirmation of single clone activity via mScarlet-I fluorescence, we used single clone sequencing to confirm that all mScarlet-I positive cells had indeed re-discovered the correct identities at positions K191, D193, and E194. These results correspond to >32,000-fold enrichment of a functional RuBisCO enzyme in a single round. Taken together, these results establish a quantitative, high-throughput strategy for RuBisCO directed evolution with a greatly diminished false-positive rate compared to traditional RDE approaches.

### Examples 7 Additional Exemplified Methods and Materials

General methods: All PCRs were performed using Phusion U HotStart DNA Polymerase (Life Technologies), Q5U Hot Start High-Fidelity DNA Polymerase (New England Biolabs), or repliQa HiFi ToughMix (Quanta Bio). Water was purified using a MilliQ water purification system (MilliporeSigma). Antibiotics (Gold Biotechnology) were used at the following concentrations for plasmid selection, unless otherwise noted: 30 μg/mL kanamycin, 40 μg/mL chloramphenicol, 50 μg/mL carbenicillin, 100 μg/mL spectinomycin, and 10 μg/mL trimethoprim. Antibiotics were used at one-third concentrations for strains bearing three unique plasmids, one-quarter concentrations for strains bearing four unique plasmids, and one-fifth concentrations for strains bearing five unique plasmids. eDRM was prepared by supplementing DRM (68) with 6.3 g/L peptone (Millipore), 0.63 g/L yeast extract (Thermo Scientific), and 22.5 mM oxalate (Combi-Blocks). We note that higher oxalate concentrations can result in significant cell death. Unless otherwise noted, all DNA assembly and plasmid propagation was performed in NEB Turbo cells (New England Biolabs) or Mach1F cells (69) (Mach1 T1^{R} cells (Thermo Fisher Scientific)) mated with F′ episome of S2060 (70)).

USER cloning: All plasmids were cloned using USER (Uracil-Specific Excision Reagent) assembly. The primers were designed to include a USER junction (spanning from a 5′ terminal dA to a dU base 13-20 base pairs downstream). USER junctions were designed to have a 30° C.<Tₘ<60° C. and minimal secondary structures. PCR products were run on a 1% agarose gel in 1× sodium borate buffer (71) containing approximately 0.2 μg/mL ethidium bromide, allowing visualization under ultraviolet light. Agarose containing PCR fragments was dissolved in Qiagen QG buffer, purified using DNA purification columns (Bio Basic), and eluted in MilliQ water. Fragments were quantified using a NanoDrop 2000 Spectrophotometer (Thermo Fisher Scientific). For USER assembly 0.1 to 0.5 pmol fragments were added in an equimolar ratio in a 10 μl reaction that included 0.75 μL USER enzyme (Uracil-DNA Glycosylase and DNA-glycosylase-lyase Endonuclease VIII; New England Biolabs), 1 ul rCutSmart Buffer (50 mM potassium acetate, 20 mM Tris-acetate, 10 mM magnesium acetate, 100 μg/mL recombinant albumen at pH 7.9; New England Biolabs) and 0.75 μL DpnI (New England Biolabs). The reactions were incubated at 37° C. for 20 min, followed by heating to 80° C. for 3 min and slow cooling to 12° C. ramped at 0.1° C./s in a thermocycler. The USER products were used for heat-shock transformation of chemically competent NEB Turbo or Mach1F cells.

Chemically competent cell preparation: To prepare competent cells, an overnight culture was diluted 100-fold in 2×YT media supplemented with maintenance antibiotics and grown at 37° C. with shaking at 350 rpm to OD₆₀₀ 0.5-0.7. Cells were pelleted by centrifugation at 5000×rcf for 5 min at 0° C. The spent media was decanted and the pellet was resuspended in the residual media by keeping it on ice for 10-15 min and gently shaking at short regular intervals. TSS (2×YT medium supplemented with 5% v/v DMSO, 10% w/v PEG 3350, and 20 mM MgCl₂) at a volume of 10% of the original culture was added to the resuspend cells and mixed by gently swirling. The cell suspension was then aliquoted, flash-frozen in liquid nitrogen, and stored at −80° C. until use.

Transformation of chemically competent cells: To transform cells, 100-400 μL of competent cells were thawed from −80° C. on ice for 15 min. An equal volume of KCM solution (100 mM KCl, 30 mM CaCl₂, 50 mM MgCl₂ in H₂O) was added to the tube and mixed gently (not vortexed). For each transformation, 2 μL of plasmid DNA (up to 3 plasmids per transformation) was added to 25 μL aliquots of competent cells/KCM mix. The mixture was incubated on ice for 10-30 min and heat-shocked at 42° C. for 90 seconds. The mixture was then chilled on ice for 2 min, then added to 1 mL of 2×YT media. Cells were allowed to recover at 37° C. with shaking at 350 RPM for at least 45 min before being streaked on 2×YT agar plates (1.5%) containing the appropriate antibiotics and incubated at 37° C. for 16-18 hours.

Bacterial genomic modifications: To delete target genes from the E. coli genome, a single DNA fragment encoding the KanR-SacB cassette was amplified using primers with 40 base pair homology regions to the gene of interest (HR1 and HR2) (72). PCR products were transformed into electrocompetent E. coli cells carrying pKD46 (73) and pre-induced with arabinose to overexpress the lambda red proteins. The mixture was allowed to recover in 2 ml of 2×YT at 30° C. overnight then struck the following morning onto solidified 2×YT+Kanamycin to select for transformants. Isolated colonies were cultured in 2×YT liquid medium with Kanamycin at 30° C. overnight. Aliquots of the overnight cultures were diluted 100-fold in water to serve as template for PCR-based genotyping. Primer pairs flanking the deleted region were used to verify mutants with target gene deletion and KanR-specific primers were used to verify KanR insertion. Clones bearing the desired deletions were then made electrocompetent and transformed with oligos encoding only the HR1 and HR2 sequences, recovered at 30° C. overnight then struck the following morning onto solidified 2×YT+5% sucrose to select loss of the KanR-SacB cassette. After 24-36 hours, single colonies were picked into 100 μl water and an aliquot of the cell suspension was used as a PCR template to verify the deletion with the same pair of flanking primers used to confirm KanR-sacB insertion. A product size transition (typically ~3 kb to ~400 bp) is indicative of the deletion, which was also confirmed by testing for sensitivity to Kan.

Adaptive Laboratory Evolution (ALE): To evolve glycolytically disrupted E. coli mutants toward optimal growth, adaptive laboratory evolution was carried out to slowly shift the media dependence of the evolving population. A starter culture of S3711 cells was used to seed a chemostat at an initial density (OD₆₀₀) of 0.05 into 250 mL of DRM^{A} supplemented with 40 mM glycerol and 40 mM succinate into duplicate chemostats. The flow rate of fresh DRM^{A}+glycerol/succinate was initially maintained at ≤0.75 vol/hr for 7 days to allow the populations to genetically drift. After this period, the DRM titration was initiated at 0.001%, and then doubled to 100% over the course of 22 days. Over the course of the ALE experiment, higher DRM concentrations required reductions of the chemostat flow rate to overcome the poor doubling time of the strain, stabilizing at 0.27 vol/hr at 100% DRM. After reaching 100% DRM, the flow rate was increased from 0.27 vol/hr to 2 vol/hr over 33 days to select for the fittest mutants. Samples from each chemostat were collected daily throughout the evolution campaign and plated on DRM^{A}+glycerol/succinate, DRM complete, and 2×YT to determine the titer of each chemostat and the relative fitness on all media.

Doubling time analysis and kinetic reporter assays: Typically, four single colonies of each strain or chemostat population were picked into 2×YT, DRM, and M9GS media and grown overnight at 37° C. with shaking at 900 RPM. Saturated cultures were diluted 50-fold into the relevant medium and 200 μl of each culture was transferred to a 96-well black wall, clear bottom plate (Costar) and overlaid with 20 ul mineral oil per well. The plate was incubated in a Spark (Tecan) plate reader running SparkControl v2.3 maintained at 37° C. with shaking (1440 rpm). Optical density was measured every 10 min over a 12-hour period and the doubling time of each well was calculated individually using GraphPad Prism (version 9).

CdaR directed evolution: To evolve CdaR toward higher specificity for glycerate, an error-prone PCR library were generated using GeneMorph II Random Mutagenesis Kit (Agilent Technologies) and cloned into a plasmid expressing a tripartite fusion protein that includes barnase, mCherry, and chloramphenicol acetyltransferase (Cat) under control of the CdaR-responsive P_{garP} promoter. The assembled library was transformed directly into S4012 and plated on 1.5% agar plates supplemented with either 2×YT or DRM alongside the maintenance antibiotics. After ~36 hours of growth, each plate was scraped to retain all genotypic diversity, diluted to a starting OD₆₀₀ of 0.05, then grown to make chemically component cells. Each population was transformed with a barnase overexpression plasmid, recovered for 3 hours at 37° C. with shaking at 350 rpm, then plated on 1.5% agar plates supplemented with either 2×YT or DRM alongside the maintenance antibiotics and 20 μg/mL chloramphenicol. After 48 hours of growth, 48 single colonies were picked from each of the four conditions overnight in 2×YT liquid medium. The following morning, each culture was diluted 50-fold into either 2×YT or DRM supplemented with the maintenance antibiotics with or without 1 mM glycerate (Millipore Sigma) in a 96-well 2 mL deep well plate, sealed with a porous film, and grown at 37° C. with shaking at 900 rpm for 24 hours. Plates were then quantified as indicated below.

Reporter assays: S4221, S4009, S4012, or S5643 cells harboring the appropriate biosensing plasmids were transformed alongside plasmids expressing RuBisCO variants and grown on 2×YT (United States Biological) with 1.5% agar supplemented with maintenance antibiotics and incubated at 37° C. overnight. Colonies were picked the following day into liquid super broth (SB) or 2×YT medium (United States Biological; SB shows improved growth over 2×YT) supplemented with the appropriate antibiotics and incubated at 37° C. with shaking at 900 rpm for 18 hours. For fluorescence assays, overnight cultures were diluted 50-fold into liquid DRM or eDRM medium (eDRM outperforms DRM for most assays) supplemented with maintenance antibiotics in a 96-well 2 mL deep well plate, sealed with a porous film, and grown at 37° C. with shaking at 900 rpm for 24 hours. In some cases, 0-100 ng/mL anhydrotetracycline (Honeywell Fluka) was added to the 96-well 2 mL deep well plate if the RuBisCO expression plasmid was under the control of the tetAp promoter. For luminescence assays, overnight cultures were diluted 50-fold into liquid DRM or eDRM medium supplemented with maintenance antibiotics in a 96-well 2 mL deep well plate, sealed with a porous film, and grown at 37° C. with shaking at 900 rpm for 4-6 hours. To quantify output, 150 μL of each culture was aliquoted into a 96-well black wall, clear bottom plate (Costar) after the appropriate incubation. OD₆₀₀ and the excitation and emission wavelengths (fluorescence) or luminescence settings were used for measurements using a Spark (Tecan) plate reader running SparkControl v2.3. Reporter signal was normalized to OD₆₀₀ after blank media subtraction. GraphPad Prism (version 9) was used for plotting and data analysis. Where relevant, non-linear dose-response functions were fitted to each variant's mScarlet-I signal to determine the EC₅₀ of the aTc titration, and Pearson correlation coefficients as well as P values were calculated in Prism. For fluorescence assays, any replicates that did not grow to an OD₆₀₀>1.0 were removed from the subsequent analysis (it is not unusual for one of the four replicates to not grow, or to grow and not fluoresce).

Diffusion assays: WT and transporter/symporter-deleted S4012 cells were transformed with plasmids encoding Prk, TtHA0368, and either a functional CbbM plasmid+mScarlet-I-based reporter (sender strain) or non-functional CbbM (K191M) plasmid+mEmerald-based reporter (receiver strain). Transformed cells were plated on 2×YT (United States Biological) with 1.5% agar supplemented with maintenance antibiotics and incubated at 37° C. overnight. Isolated colonies were picked into SB medium supplemented with maintenance antibiotics and grown at 37° C. with shaking at 350 rpm overnight. Overnight cultures were diluted 50-fold into eDRM supplemented with maintenance antibiotics in a 96-well 2 mL deep well plate and grown at 37° C. with shaking at 900 rpm overnight. For each diffusion assay between plated culture, 1 uL of overnight DRM cultures were spotted on a DRM plate supplemented with maintenance antibiotics and incubated at 37° C. For the glycerate diffusion assay, 1 uL of 50 mM glycerate was spotted adjacent to 1 uL spots of saturated receiver cultures on an eDRM plate supplemented with maintenance antibiotics and incubated at 37° C. Each array of spots contains one spot of the donor strain (or glycerate) at the top left-hand corner and 3 biological replicates of a receiver strain. The fluorescence images were taken using an Azure c400 Imaging system after 48 hours of incubation.

Mock evolution: An NNK degenerate library randomizing all CbbM essential residues K191, D193 and E194 was created by PCR using primers synthesized to be degenerate at those positions. The PCR was performed using the inactive template CbbM (K191M) to minimize contamination in downstream steps. Next, this fragment library was USER-assembled and transformed into S5643 competent cells harboring TtHA0368, PrkA(R52A), and CdaR^{evo1}. Transformants were plated on 2×YT (United States Biological) with 1.5% agar supplemented with maintenance antibiotics and incubated at 37° C. overnight. After 24 hours of growth, all resultant colonies were pooled by scraping, resuspended in eDRM liquid medium, diluted to starting OD₆₀₀ of 0.05, and incubated overnight at 37° C. with 5% CO₂. After 24 hours of growth, the library culture was washed with PBS buffer three times successively then diluted 100-fold in preparation for fluorescence activated cell sorting (FACS). Analysis was carried out using a Beckman Coulter Astrios, with excitation/emission wavelengths of 561/614 nm and 488/513 nm for mScarlet-I and sfGFP, respectively. The sorting gate was chosen to select for intact cells within the top 5% of fluorescence values for mScarlet-I. Collected cells were immediately plated on 2×YT with 1.5% agar supplemented with maintenance antibiotics and incubated at 37° C. overnight. Cultures were used to inoculate an mScarlet-I fluorescence assay as described above. Following confirmation of positive mScarlet-I signal, RuBisCO plasmids from functional cultures were purified and subjected to Sanger sequencing.

Computational analyses: Structure predictions of T. thermus HA0368, E. coli CdaR, and R. rubrum CA were made using Alphafold (74, 75) run on Google Collaboratory (76). Protein-ligand interactions were predicted using CB-Dock (77). The phylogenetic tree was constructed using Clustal Omega (78) multiple sequence alignment and iTOL (79) for visualization.

Additional information on a number of vectors, various modified genes, and genotypes of bacterial strains exemplified herein are provided in Tables 1-4 below.

TABLE 1
Order of Gene Deletion to Create an Insulated RuBisCO-Biosensing E. coli
(iRBE) Strain. In some cases, contiguous operons were deleted to minimize
the biosynthesis of metabolite-specific enzymes not included in FIG.
6. To simplify the number of recombineering steps, the ΔallS-allD
cassette (which includes many non-essential bystander genes that are
not relevant to any metabolite) was deleted in a single step.

| | | | | | | Nearest |
|---|---|---|---|---|---|---|
| | | | Start | End | Length | Deletion |
| Order | Metabolite | Gene Name | (bp) | (bp) | (bp) | (bp) |
| 1 | 3- | ΔserA | 3,149,070 | 3,150,302 | 1,232 | 13,049 |
| | Phosphoglycerate | | | | | |
| 2 | 3- | Δeda-edd | 2,020,710 | 2,023,199 | 2,489 | 68,349 |
| | Phosphoglycerate | | | | | |
| 3 | Glycerate | ΔgarDPLRK | 3,366,392 | 3,372,620 | 6,228 | 141,589 |
| 4 | Glycolate | ΔglcCDEFGB | 3,217,401 | 3,224,803 | 7,402 | 52,887 |
| 5 | Glycerate | ΔgudPXD | 3,009,945 | 3,014,000 | 4,055 | 11,440 |
| 6 | Glycerate/ | ΔallS-allD | 469,850 | 484,918 | 15,068 | 233,766 |
| | Glycolate | (+glxK, glxR, | | | | |
| | | hyi) | | | | |
| 7 | Glycerate | ΔcdaR | 156,567 | 157,724 | 1,157 | 163,775 |
| 8 | Glycerate/ | ΔghrA | 1,152,554 | 1,153,492 | 938 | 2,027 |
| | Glycolate | | | | | |
| 9 | Glycerate/ | ΔghrB | 3,812,910 | 3,813,884 | 974 | 66,976 |
| | Glycolate | | | | | |
| 10 | 3- | ΔytjC | 4,678,282 | 4,678,929 | 647 | 163,775 |
| | Phosphoglycerate | | | | | |
| 11 | 3- | Δpgk | 3,163,351 | 3,164,514 | 1,163 | 13,049 |
| | Phosphoglycerate | | | | | |
| 12 | 3- | ΔgpmA | 838,658 | 839,410 | 752 | 300,262 |
| | Phosphoglycerate | | | | | |
| 13 | 3- | ΔgpmM | 3,880,860 | 3,882,404 | 1,544 | 35,303 |
| | Phosphoglycerate | | | | | |
| 14 | 3- | ΔgapA | 1,951,366 | 1,952,361 | 995 | 68,349 |
| | Phosphoglycerate | | | | | |
| 15 | Glycolate | ΔaceBAK | 4,313,197 | 4,318,051 | 4,854 | 360,231 |
| 16 | 2- | Δgph | 3,609,398 | 3,610,156 | 758 | 202,754 |
| | Phosphoglycolate | | | | | |

TABLE 2
Genotypes of Key Bacterial Strains Used in this Work.

| Strain | Condensed | |
|---|---|---|
| Number | Genotype | Full Genotype |
| S1021 | S1021 | F− λ− endA1 recA1 galE15 galK16 nupG rpsL ΔlacIZYA araD139 |
| | | Δ(ara, leu)7697 mcrA Δ(mrr-hsdRMS-mcrBC) proBA::pir116 araE201 ΔrpoZ |
| | | Δflu ΔcsgABCDEFG ΔpgaC |
| S3573 | S3573 | F− λ− endA1 recA1 galE15 galK16 nupG rpsL ΔlacIZYA araD139 |
| | Δpgk | Δ(ara, leu)7697 mcrA Δ(mrr-hsdRMS-mcrBC) proBA::pir116 araE201 ΔrpoZ |
| | | Δflu ΔcsgABCDEFG ΔpgaC |
| | | lptD(V430I) dcm(A372T) ygfK(G757D) rbsB(E271*) ΔserA Δeda-edd |
| | | ΔgarDPLRK ΔglcCDEFGB ΔgudPXD ΔallS-allD ΔglxK ΔglxR Δhyi ΔcdaR |
| | | ΔghrA ΔghrB ΔytjC |
| S3711 | S3573 | F− λ− endA1 recA1 galE15 galK16 nupG rpsL ΔlacIZYA araD139 |
| | Δpgk | Δ(ara, leu)7697 mcrA Δ(mrr-hsdRMS-mcrBC) proBA::pir116 araE201 ΔrpoZ |
| | | Δflu ΔcsgABCDEFG ΔpgaC |
| | | lptD(V430I) dcm(A372T) ygfK(G757D) rbsB(E271*) ΔserA Δeda-edd |
| | | ΔgarDPLRK ΔglcCDEFGB ΔgudPXD ΔallS-allD ΔglxK ΔglxR Δhyi ΔcdaR |
| | | ΔghrA ΔghrB ΔytjC |
| S3926 | — | F+ (pOX38^{int} = pOX38-Tc::lacI^{Q1} P_{N25} -tetR luxCDE Pₚₛₚ (AR2) lacZ, |
| | | integrated in PifA) λ− rph-1 |
| S4003 | — | F+ λ− endA1 recA1 galE15 galK16 nupG rpsL ΔlacIZYA araD139 |
| | | Δ(ara, leu)7697 mcrA Δ(mrr-hsdRMS-mcrBC) proBA::pir116 araE201 ΔrpoZ |
| | | Δflu ΔcsgABCDEFG ΔpgaC |
| | | lptD(V430I) dcm(A372T) ygfK(G757D) rbsB(E271*) ΔserA Δeda-edd |
| | | ΔgarDPLRK ΔglcCDEFGB ΔgudPXD ΔallS-allD ΔglxK ΔglxR Δhyi ΔcdaR |
| | | ΔghrA ΔghrB ΔytjC |
| | | Δpgk fabH(A216V) rbn(V119I) recA(D161G) dtpB(F97L) ΔgpmA ΔgpmM |
| | | ΔgapA ΔaceBAK Δgph mgsA(V111A) mhpT(T37A) nrdR(A44V) |
| | | rhsD(W159fs) lnt(F498L) |
| | | rhsC(Y1167C) mukB(G182D) rutD(H221D) ptsG(P154L) adhE(V6fs) |
| | | ydcI(D160G) ydcV(T84A) ydeQ(A224T) ydfR(P115L) manX(T12*) |
| | | yodD(V15A) yehC(V13A) |
| | | ccmA(I178fs) yfbV(P52L) prmB(T235A) ypfG(F11fs) nrdE(L665fs) |
| | | syd(T122A) lysA(G354E) xdhA(A331fs) yghQ(V78A) rpsG(V141I) |
| | | tusD(T108A) rhs(K74fs) |
| S4006 | — | F− λ− endA1 recA1 galE15 galK16 nupG rpsL ΔlacIZYA araD139 |
| | | Δ(ara, leu)7697 mcrA Δ(mrr-hsdRMS-mcrBC) proBA::pir116 araE201 ΔrpoZ |
| | | Δflu ΔcsgABCDEFG ΔpgaC |
| | | lptD(V430I) dcm(A372T) ygfK(G757D) rbsB(E271*) ΔserA Δeda-edd |
| | | ΔgarDPLRK ΔglcCDEFGB ΔgudPXD ΔallS-allD ΔglxK ΔglxR Δhyi ΔcdaR |
| | | ΔghrA ΔghrB ΔytjC |
| | | Δpgk fabH(A216V) rbn(V119I) recA(D161G) dtpB(F97L) ΔgpmA ΔgpmM |
| | | ΔgapA ΔaceBAK Δgph mgsA(S79P) ydiY(G102fs) yeiW(W74_L78del) |
| | | rhaM(V67A) |
| S4221 | S3573 X | F+ (pOX38^{int}) λ− endA1 recA1 galE15 galK16 nupG rpsL ΔlacIZYA araD139 |
| | S3926 | Δ(ara, leu)7697 mcrA Δ(mrr-hsdRMS-mcrBC) proBA::pir116 araE201 ΔrpoZ |
| | | Δflu |
| | | ΔcsgABCDEFG ΔpgaC lptD(V430I) dcm(A372T) ygfK(G757D) rbsB(E271*) |
| | | ΔserA Δeda-edd ΔgarDPLRK ΔglcCDEFGB ΔgudPXD ΔallS-allD ΔglxK |
| | | ΔglxR Δhyi |
| S4009 | S4003 X | F+ (pOX38^{int}) λ− endA1 recA1 galE15 galK16 nupG rpsL ΔlacIZYA araD139 |
| | S3926 | Δ(ara, leu)7697 mcrA Δ(mrr-hsdRMS-mcrBC) proBA::pir116 araE201 ΔrpoZ |
| | | Δflu |
| | | ΔcsgABCDEFG ΔpgaC lptD(V430I) dcm(A372T) ygfK(G757D) rbsB(E271*) |
| | | ΔserA Δeda-edd ΔgarDPLRK ΔglcCDEFGB ΔgudPXD ΔallS-allD ΔglxK |
| | | ΔglxR Δhyi |
| | | ΔcdaR ΔghrA ΔghrB ΔytjC Δpgk fabH(A216V) rbn(V119I) recA(D161G) |
| | | dtpB(F97L) ΔgpmA ΔgpmM ΔgapA ΔaceBAK Δgph mgsA(V111A) |
| | | mhpT(T37A) nrdR(A44V) |
| | | rhsD(W159fs) lnt(F498L) rhsC(Y1167C) mukB(G182D) rutD(H221D) |
| | | ptsG(P154L) adhE(V6fs) ydcI(D160G) ydcV(T84A) ydeQ(A224T) |
| | | ydfR(P115L) manX(T12*) |
| | | yodD(V15A) yehC(V13A) ccmA(I178fs) yfbV(P52L) prmB(T235A) |
| | | ypfG(F11fs) nrdE(L665fs) syd(T122A) lysA(G354E) xdhA(A331fs) |
| | | yghQ(V78A) rpsG(V141I) |
| S4012 | S4006 X | F+ (pOX38^{int}) λ− endA1 recA1 galE15 galK16 nupG rpsL ΔlacIZYA araD139 |
| | S3926 | Δ(ara, leu)7697 mcrA Δ(mrr-hsdRMS-mcrBC) proBA::pir116 araE201 ΔrpoZ |
| | | Δflu |
| | | ΔcsgABCDEFG ΔpgaC lptD(V430I) dcm(A372T) ygfK(G757D) rbsB(E271*) |
| | | ΔserA Δeda-edd ΔgarDPLRK ΔglcCDEFGB ΔgudPXD ΔallS-allD ΔglxK |
| | | ΔglxR Δhyi |
| | | ΔcdaR ΔghrA ΔghrB ΔytjC Δpgk fabH(A216V) rbn(V119I) recA(D161G) |
| | | dtpB(F97L) ΔgpmA ΔgpmM ΔgapA ΔaceBAK Δgph mgsA(S79P) |
| | | ydiY(G102fs) |
| S5628 | S4012 | F+ (pOX38^{int}) λ− endA1 recA1 galE15 galK16 nupG rpsL ΔlacIZYA |
| | ΔdauA | araD139 Δ(ara, leu)7697 mcrA Δ(mrr-hsdRMS-mcrBC) proBA::pir116 |
| | | araE201 ΔrpoZ Δflu |
| | | ΔcsgABCDEFG ΔpgaC lptD(V430I) dcm(A372T) ygfK(G757D) rbsB(E271*) |
| | | ΔserA Δeda-edd ΔgarDPLRK ΔglcCDEFGB ΔgudPXD ΔallS-allD ΔglxK |
| | | ΔglxR Δhyi |
| | | ΔcdaR ΔghrA ΔghrB ΔytjC Δpgk fabH(A216V) rbn(V119I) recA(D161G) |
| | | dtpB(F97L) ΔgpmA ΔgpmM ΔgapA ΔaceBAK Δgph mgsA(S79P) |
| | | ydiY(G102fs) |
| S5629 | S4012 | F+ (pOX38^{int}) λ− endA1 recA1 galE15 galK16 nupG rpsL ΔlacIZYA |
| | ΔglcA | araD139 Δ(ara, leu)7697 mcrA Δ(mrr-hsdRMS-mcrBC) proBA::pir116 |
| | | araE201 ΔrpoZ Δflu |
| | | ΔcsgABCDEFG ΔpgaC lptD(V430I) dcm(A372T) ygfK(G757D) rbsB(E271*) |
| | | ΔserA Δeda-edd ΔgarDPLRK ΔglcCDEFGB ΔgudPXD ΔallS-allD ΔglxK |
| | | ΔglxR Δhyi |
| | | ΔcdaR ΔghrA ΔghrB ΔytjC Δpgk fabH(A216V) rbn(V119I) recA(D161G) |
| | | dtpB(F97L) ΔgpmA ΔgpmM ΔgapA ΔaceBAK Δgph mgsA(S79P) |
| | | ydiY(G102fs) |
| S5630 | S4012 | F+ (pOX38^{int}) λ− endA1 recA1 galE15 galK16 nupG rpsL ΔlacIZYA |
| | ΔdctA | araD139 Δ(ara, leu)7697 mcrA Δ(mrr-hsdRMS-mcrBC) proBA::pir116 |
| | | araE201 ΔrpoZ Δflu |
| | | ΔcsgABCDEFG ΔpgaC lptD(V430I) dcm(A372T) ygfK(G757D) rbsB(E271*) |
| | | ΔserA Δeda-edd ΔgarDPLRK ΔglcCDEFGB ΔgudPXD ΔallS-allD ΔglxK |
| | | ΔglxR Δhyi |
| | | ΔcdaR ΔghrA ΔghrB ΔytjC Δpgk fabH(A216V) rbn(V119I) recA(D161G) |
| | | dtpB(F97L) ΔgpmA ΔgpmM ΔgapA ΔaceBAK Δgph mgsA(S79P) |
| | | ydiY(G102fs) |
| S5632 | ΔlldP::kan | F+ (pOX38^{int}) λ− endA1 recA1 galE15 galK16 nupG rpsL ΔlacIZYA |
| | | araD139 Δ(ara, leu)7697 mcrA Δ(mrr-hsdRMS-mcrBC) proBA::pir116 |
| | | araE201 ΔrpoZ Δflu |
| | | ΔcsgABCDEFG ΔpgaC lptD(V430I) dcm(A372T) ygfK(G757D) rbsB(E271*) |
| | | ΔserA Δeda-edd ΔgarDPLRK ΔglcCDEFGB ΔgudPXD ΔallS-allD ΔglxK |
| | | ΔglxR Δhyi |
| | | ΔcdaR ΔghrA ΔghrB ΔytjC Δpgk fabH(A216V) rbn(V119I) recA(D161G) |
| | | dtpB(F97L) ΔgpmA ΔgpmM ΔgapA ΔaceBAK Δgph mgsA(S79P) |
| | | ydiY(G102fs) |
| S5633 | S4012 | F+ (pOX38^{int}) λ− endA1 recA1 galE15 galK16 nupG rpsL ΔlacIZYA |
| | ΔactP | araD139 Δ(ara, leu)7697 mcrA Δ(mrr-hsdRMS-mcrBC) proBA::pir116 |
| | | araE201 ΔrpoZ Δflu |
| | | ΔcsgABCDEFG ΔpgaC lptD(V430I) dcm(A372T) ygfK(G757D) rbsB(E271*) |
| | | ΔserA Δeda-edd ΔgarDPLRK ΔglcCDEFGB ΔgudPXD ΔallS-allD ΔglxK |
| | | ΔglxR Δhyi |
| | | ΔcdaR ΔghrA ΔghrB ΔytjC Δpgk fabH(A216V) rbn(V119I) recA(D161G) |
| | | dtpB(F97L) ΔgpmA ΔgpmM ΔgapA ΔaceBAK Δgph mgsA(S79P) |
| | | ydiY(G102fs) |
| S5634 | S4012 | F+ (pOX38^{int}) λ− endA1 recA1 galE15 galK16 nupG rpsL ΔlacIZYA |
| | ΔyaaH | araD139 Δ(ara, leu)7697 mcrA Δ(mrr-hsdRMS-mcrBC) proBA::pir116 |
| | | araE201 ΔrpoZ Δflu |
| | | ΔcsgABCDEFG ΔpgaC lptD(V430I) dcm(A372T) ygfK(G757D) rbsB(E271*) |
| | | ΔserA Δeda-edd ΔgarDPLRK ΔglcCDEFGB ΔgudPXD ΔallS-allD ΔglxK |
| | | ΔglxR Δhyi |
| | | ΔcdaR ΔghrA ΔghrB ΔytjC Δpgk fabH(A216V) rbn(V119I) recA(D161G) |
| | | dtpB(F97L) ΔgpmA ΔgpmM ΔgapA ΔaceBAK Δgph mgsA(S79P) |
| | | ydiY(G102fs) |

TABLE 3
Putative Glycerate Transporters. Transporters were
nominated by their ability to transport molecules
with structural of chemical similarity to glycerate.

| | | Reference Position | |
|---|---|---|---|
| Gene | Activity | (bp, DH10B) | GenBank |
| dauA | Aerobic C4- | 1259124 . . . 1260803 | NC_000913.3 |
| | dicarboxylate | | |
| | transporter | | |
| glcA | Glycolate/lactate:H(+) | 3119597 . . . 3121279 | NC_000913.3 |
| | symporter | | |
| dctA | C4 | 3682161 . . . 3683447 | NC_000913.3 |
| | dicarboxylate/oro- | | |
| | tate:H(+) symporter | | |
| lldP | Lactate/glycolate:H(+) | 3777399 . . . 3779054 | NC_000913.3 |
| | symporter | | |
| actP | Acetate/glycolate:cation | 4283253 . . . 4284902 | NC_000913.3 |
| | symporter | | |
| yaaH | Acetate/succinate:H(+) | 9928 . . . 10494 | NC_000913.3 |
| | symporter | | |

TABLE 4
Key Proteins and Enzymes Used in this Work. In all cases, accession numbers and relevant mutations explored in this study have been included.

| Circuit | | | Source | | | NCBI | Relevant |
|---|---|---|---|---|---|---|---|
| Function | Name | Activity | Organism | UniProt | GenBank | RefSeq | Mutations |
| Transcriptional | CdaR | Carbohydrate | E. coli | P37047 | AAC73273.2 | — | R20C, |
| Activator | | diacid | | | | | I21F, |
| | | regulator | | | | | G50C, |
| | | | | | | | C109G, |
| | | | | | | | C109R, |
| | | | | | | | Q118H, |
| | | | | | | | M122K |
| 3PG | StPgtP | Phosphoglycerate | S. typhimurium | P12681 | AAL21299.1 | — | |
| Import | | transporter | | | | | |
| | | protein | | | | | |
| 3PG | EcUhpT | Hexose-6- | E. coli | P0AGC0 | AAC76689.1 | | D388C |
| Import | | phosphate:phosphate | | | | | |
| | | antiporter | | | | | |
| 3PG De- | Gph | Phosphoglycolate | E. coli | P32662 | AAC76410.1 | — | |
| phosphorylation | | phosphatase | | | | | |
| 3PG De- | YieH | 6- | E. coli | P31467 | AAC76738.1 | — | |
| phosphorylation | | phosphogluconate | | | | | |
| | | phosphatase | | | | | |
| 3PG De- | YbhA | Pyridoxal | E. coli | P21829 | AAC73853.1 | — | |
| phosphorylation | | phosphate | | | | | |
| | | phosphatase | | | | | |
| 3PG De- | ScPho13 | 4- | S. cerevisiae | P19881 | DAA11630.1 | — | |
| phosphorylation | | nitrophenylphosphatase | | | | | |
| 3PG De- | ScYOR283w | phosphoglycerate | S. cerevisiae | Q12040 | DAA11047.1 | — | |
| phosphorylation | | mutase | | | | | |
| 3PG De- | TtHA0368 | Phosphoglycerate | T. thermophilus | Q5SLC5 | — | WP_008634004.1 | R8A, |
| phosphorylation | | mutase | | | | | H9A, |
| | | | | | | | R60A, |
| | | | | | | | E84A, |
| | | | | | | | H148A |
| 3PG De- | GsPhoE | Phosphoglycerate | G. stearothermophilus | Q9ALU0 | AF343668.1 | — | |
| phosphorylation | | mutase | | | | | |
| Ribulose-5- | PrkA | Phosphoribulokinase | Synechococcus | Q31PL2 | — | — | K20A, |
| phosphate | | | elongatus | | | | S21A, |
| phosphorylation | | | | | | | T22A, |
| | | | | | | | H45A, |
| | | | | | | | R49A, |
| | | | | | | | R52A, |
| | | | | | | | Y88L, |
| | | | | | | | E110A, |
| | | | | | | | W140A, |
| | | | | | | | R163A, |
| | | | | | | | T304A |
| 3PG | RrCbbM | Ribulose | Rhodospirillum | P04718 | CAA25080.1 | — | K191M, |
| Production | | bisphosphate | rubrum | | | | D193N, |
| | | carboxylase | | | | | E194Q |

### REFERENCES

1. Y. M. Bar-On, R. Milo, The global mass and average rate of rubisco. Proc Natl Acad Sci USA 116, 4738-4743 (2019).
2. C. Beer et al., Terrestrial gross carbon dioxide uptake: global distribution and covariation with climate. Science 329, 834-838 (2010).
3. A. Bar-Even et al., The moderately efficient enzyme: evolutionary and physicochemical trends shaping enzyme parameters. Biochemistry 50, 4402-4410 (2011).
4. B. J. Walker, A. VanLoocke, C. J. Bernacchi, D. R. Ort, The Costs of Photorespiration to Food Production Now and in the Future. Annual Review of Plant Biology 67, 107-129 (2016).
5. R. Kebeish et al., Chloroplastic photorespiratory bypass increases photosynthesis and biomass production in Arabidopsis thaliana. Nat Biotechnol 25, 593-599 (2007).
6. S. P. Long, A. Marshall-Colon, X. G. Zhu, Meeting the global food demand of the future by engineering crop photosynthesis and yield potential. Cell 161, 56-66 (2015).
7. R. J. Ellis, The most abundant protein in the world. Trends in Biochemical Sciences, (1979).
8. D. Davidi et al., Highly active rubiscos discovered by systematic interrogation of natural sequence diversity. EMBO J 39, e104081 (2020).
9. M. T. Lin, H. Salihovic, F. K. Clark, M. R. Hanson, Improving the efficiency of Rubisco by resurrecting its ancestors in the family Solanaceae. Sci Adv 8, eabm6871 (2022).
10. A. K. Liu et al., Structural plasticity enables evolution and innovation of RuBisCO assemblies. Sci Adv 8, eadc9440 (2022).
11. L. Schulz et al., Evolution of increased complexity and specificity at the dawn of form I Rubiscos. Science 378, 155-160 (2022).
12. D. N. Greene, S. M. Whitney, I. Matsumura, Artificially evolved Synechococcus PCC6301 Rubisco variants exhibit improvements in folding and catalytic efficiency. Biochem J 404, 517-524 (2007).
13. Y. Zhou, S. Whitney, Directed Evolution of an Improved Rubisco; In Vitro Analyses to Decipher Fact from Fiction. Int J Mol Sci 20, (2019).
14. R. H. Wilson, E. Martin-Avila, C. Conlan, S. M. Whitney, An improved Escherichia coli screen for Rubisco identifies a protein-protein interface that can enhance CO2-fixation kinetics. J Biol Chem 293, 18-27 (2018).
15. S. Satagopan, K. A. Huening, F. R. Tabita, Selection of Cyanobacterial (Synechococcus sp. Strain PCC 6301) RubisCO Variants with Improved Functional Properties That Confer Enhanced C02-Dependent Growth of Rhodobacter capsulatus, a Photosynthetic Bacterium. mBio 10, (2019).
16. S. Gleizer et al., Conversion of Escherichia coli to Generate All Biomass Carbon from CO2. Cell 179, 1255-1263 e1212 (2019).
17. O. Mueller-Cajar, M. Morell, S. M. Whitney, Directed evolution of Rubisco in Escherichia coli reveals a specificity-determining hydrogen bond in the form II enzyme. Biochemistry 46, 14067-14074 (2007).
18. M. R. Parikh, D. N. Greene, K. K. Woods, I. Matsumura, Directed evolution of RuBisCO hypermorphs through genetic selection in engineered E. coli. Protein Eng Des Sel 19, 113-119 (2006).
19. D. Szklarczyk et al., STRING v11: protein-protein association networks with increased coverage, supporting functional discovery in genome-wide experimental datasets. Nucleic Acids Res 47, D607-D613 (2019).
20. I. M. Keseler et al., The EcoCyc Database in 2021. Front Microbiol 12, 711077 (2021).
21. T. Baba et al., Construction of Escherichia coli K-12 in-frame, single-gene knockout mutants: the Keio collection. Mol Syst Biol 2, 2006 0008 (2006).
22. J. C. Carlson, A. H. Badran, D. A. Guggiana-Nilo, D. R. Liu, Negative selection and stringency modulation in phage-assisted continuous evolution. Nat Chem Biol 10, 216-222 (2014).
23. F. Liu, S. Bratulic, A. Costello, T. P. Miettinen, A. H. Badran, Directed evolution of rRNA improves translation kinetics and recombinant protein yield. Nat Commun 12, 5638 (2021).
24. M. H. Irani, P. K. Maitra, Properties of Escherichia coli mutants deficient in enzymes of glycolysis. J Bacteriol 132, 398-410 (1977).
25. M. Irani, P. K. Maitra, Isolation and characterization of Escherichia coli mutants defective in enzymes of glycolysis. Biochem Biophys Res Commun 56, 127-133 (1974).
26. A. Wellner, M. Raitses Gurevich, D. S. Tawfik, Mechanisms of protein sequence divergence and incompatibility. Plos Genet 9, e1003665 (2013).
27. T. W. Lo, M. E. Westwood, A. C. McLellan, T. Selwood, P. J. Thornalley, Binding and modification of proteins by methylglyoxal under physiological conditions. A kinetic and mechanistic study with N alpha-acetylarginine, N alpha-acetylcysteine, and N alpha-acetyllysine, and bovine serum albumin. J Biol Chem 269, 32299-32305 (1994).
28. S. Totemeyer, N. A. Booth, W. W. Nichols, B. Dunbar, I. R. Booth, From famine to feast: the role of methylglyoxal production in Escherichia coli. Mol Microbiol 27, 553-562 (1998).
29. H. Falahati et al., Transmitting the allosteric signal in methylglyoxal synthase. Protein Eng Des Sel 26, 445-452 (2013).
30. C. Iacometti et al., Activating Silent Glycolysis Bypasses in <i>Escherichia coli</i>. BioDesign Research 2022, U.S. Pat. No. 9,859,643 (2022).
31. K. G. Anthony, C. Sherburne, R. Sherburne, L. S. Frost, The role of the pilus in recipient cell recognition during bacterial conjugation mediated by F-like plasmids. Mol Microbiol 13, 939-953 (1994).
32. B. P. Hubbard et al., Continuous directed evolution of DNA-binding proteins to improve TALEN specificity. Nat Methods 12, 939-942 (2015).
33. R. Monterrubio, L. Baldoma, N. Obradors, J. Aguilar, J. Badia, A common regulator for the operons encoding the enzymes involved in D-galactarate, D-glucarate, and D-glycerate utilization in Escherichia coli. J Bacteriol 182, 2672-2674 (2000).
34. M. M. Sampaio et al., Phosphotransferase-mediated transport of the osmolyte 2-O-alpha-mannosyl-D-glycerate in Escherichia coli occurs by the product of the mngA (hrsA) gene and is regulated by the mngR (farR) gene product acting as repressor. J Biol Chem 279, 5537-5548 (2004).
35. A. B. Cubitt, L. A. Woollenweber, R. Heim, Understanding structure-function relationships in the Aequorea victoria green fluorescent protein. Methods Cell Biol 58, 19-30 (1999).
36. D. S. Bindels et al., mScarlet: a bright monomeric red fluorescent protein for cellular imaging. Nat Methods 14, 53-56 (2017).
37. N. S. Kolber, R. Fattal, S. Bratulic, G. D. Carver, A. H. Badran, Orthogonal translation enables heterologous ribosome engineering in E. coli. Nature Communications 12, 599 (2021).
38. S. Q. Jiang, G. Q. Yu, Z. G. Li, J. S. Hong, Genetic evidence for modulation of the activator by two regulatory proteins involved in the exogenous induction of phosphoglycerate transport in Salmonella typhimurium. J Bacteriol 170, 4304-4308 (1988).
39. J. A. Hall, M. C. Fann, P. C. Maloney, Altered substrate selectivity in a mutant of an intrahelical salt bridge in UhpT, the sugar phosphate carrier of Escherichia coli. J Biol Chem 274, 6148-6153 (1999).
40. E. Kuznetsova et al., Genome-wide analysis of substrate specificities of the Escherichia coli haloacid dehalogenase-like phosphatase family. J Biol Chem 281, 36149-36161 (2006).
41. E. Kuznetsova et al., Functional Diversity of Haloacid Dehalogenase Superfamily Phosphatases from Saccharomyces cerevisiae: BIOCHEMICAL, STRUCTURAL, AND EVOLUTIONARY INSIGHTS. J Biol Chem 290, 18678-18698 (2015).
42. C. K. Ho, A. F. Lam, L. S. Symington, Identification of nucleases and phosphatases by direct biochemical screen of the Saccharomyces cerevisiae proteome. PLoS One 4, e6993 (2009).
43. Y. Chiba, K. Oshima, H. Arai, M. Ishii, Y. Igarashi, Discovery and analysis of cofactor-dependent phosphoglycerate mutase homologs as novel phosphoserine phosphatases in Hydrogenobacter thermophilus. J Biol Chem 287, 11934-11941 (2012).
44. A. Khlebnikov, K. A. Datsenko, T. Skaug, B. L. Wanner, J. D. Keasling, Homogeneous expression of the P(BAD) promoter in Escherichia coli by constitutive expression of the low-affinity high-capacity AraE transporter. Microbiology (Reading) 147, 3241-3247 (2001).
45. C. M. Thomas, M. A. Cross, A. A. Hussain, C. A. Smith, Analysis of copy number control elements in the region of the vegetative replication origin of the broad host range plasmid RK2. EMBO J 3, 57-63 (1984).
46. A. Haldimann, L. L. Daniels, B. L. Wanner, Use of new methods for construction of tightly regulated arabinose and rhamnose promoter fusions in studies of the Escherichia coli phosphate regulon. Journal of Bacteriology 180, 1277-1286 (1998).
47. S. Ringquist et al., Translation Initiation in Escherichia-coli—Sequences within the Ribosome-Binding Site. Mol Microbiol 6, 1219-1229 (1992).
48. A. Yu et al., Photosynthetic Phosphoribulokinase Structures: Enzymatic Mechanisms and the Redox Regulation of the Calvin-Benson-Bassham Cycle. Plant Cell 32, 1556-1573 (2020).
49. N. Antonovsky et al., Sugar Synthesis from CO2 in Escherichia coli. Cell 166, 115-125 (2016).
50. L. W. Tari, A. Matte, U. Pugazhenthi, H. Goldie, L. T. Delbaere, Snapshot of an enzyme reaction intermediate in the structure of the ATP-Mg2+-oxalate ternary complex of Escherichia coli PEP carboxykinase. Nat Struct Biol 3, 355-363 (1996).
51. D. Davidi et al., Highly active rubiscos discovered by systematic interrogation of natural sequence diversity. Embo Journal 39, (2020).
52. S. Cabantous, T. C. Terwilliger, G. S. Waldo, Protein tagging and detection with engineered self-assembling fragments of green fluorescent protein. Nat Biotechnol 23, 102-107 (2005).
53. I. Andersson et al., Crystal-Structure of the Active-Site of Ribulose-Bisphosphate Carboxylase. Nature 337, 229-234 (1989).
54. C. C. Mann, Genetic Engineers Aim to Soup Up Crop Photosynthesis. Science 283, 314-316 (1999).
55. R. J. Spreitzer, Genetic Dissection of Rubisco Structure and Function. Annual Review of Plant Physiology and Plant Molecular Biology 44, 411-434 (1993).
56. C. R. Somerville, W. L. Ogren, A phosphoglycolate phosphatase-deficient mutant of Arabidopsis. Nature 280, 833-836 (1979).
57. M. K. Morell, K. Paul, H. J. Kane, T. J. Andrews, Rubisco—Maladapted or Misunderstood. Aust J Bot 40, 431-441 (1992).
58. E. Herz et al., The genetic basis for the adaptation of E. coli to sugar synthesis from CO2. Nature Communications 8, 1705 (2017).
59. Directed Enzyme Evolution: Screening and Selection Methods. (Humana Totowa, NJ, 2003).
60. C. Iacometti et al., Activating Silent Glycolysis Bypasses in Escherichia coli. BioDesign Research 2022, 1-17 (2022).
61. D. Saadat, D. H. Harrison, The crystal structure of methylglyoxal synthase from Escherichia coli. Structure 7, 309-317 (1999).
62. M. Varadi et al., AlphaFold Protein Structure Database: massively expanding the structural coverage of protein-sequence space with high-accuracy models. Nucleic Acids Research 50, D439-D444 (2021).
63. J. Jumper et al., Highly accurate protein structure prediction with AlphaFold. Nature 596, 583-589 (2021).
64. Y. Liu et al., CB-Dock: a web server for cavity detection-guided protein-ligand blind docking. Acta Pharmacologica Sinica 41, 138-144 (2020).
65. Y. Cao, L. Li, Improved protein-ligand binding affinity prediction by using a curvature-dependent surface-area model. Bioinformatics 30, 1674-1680 (2014).
66. S. Ringquist et al., Translation initiation in Escherichia coli: sequences within the ribosome-binding site. Molecular Microbiology 6, 1219-1229 (1992).
67. M. J. Giacalone et al., Toxic protein expression in Escherichia coli using a rhamnose-based tightly regulated and tunable promoter system. Biotechniques 40, 355-364 (2006).
68. J. C. Carlson, A. H. Badran, D. A. Guggiana-Nilo, D. R. Liu, Negative selection and stringency modulation in phage-assisted continuous evolution. Nature Chemical Biology 10, 216-222 (2014).
69. N. S. Kolber, R. Fattal, S. Bratulic, G. D. Carver, A. H. Badran, Orthogonal translation enables heterologous ribosome engineering in E. coli. Nature Communications 12, 599 (2021).
70. B. P. Hubbard et al., Continuous directed evolution of DNA-binding proteins to improve TALEN specificity. Nature Methods 12, 939-942 (2015).
71. J. R. Brody, S. E. Kern, Sodium boric acid: a Tris-free, cooler conductive medium for DNA electrophoresis. Biotechniques 36, 214-216 (2004).
72. F. Liu, S. Bratulic, A. Costello, T. P. Miettinen, A. H. Badran, Directed evolution of rRNA improves translation kinetics and recombinant protein yield. Nat Commun 12, 5638 (2021).
73. K. A. Datsenko, B. L. Wanner, One-step inactivation of chromosomal genes in Escherichia coli K-12 using PCR products. Proc Natl Acad Sci USA 97, 6640-6645 (2000).
74. J. Jumper et al., Highly accurate protein structure prediction with AlphaFold. Nature 596, 583-589 (2021).
75. M. Varadi et al., AlphaFold Protein Structure Database: massively expanding the structural coverage of protein-sequence space with high-accuracy models. Nucleic Acids Research 50, D439-D444 (2021).
76. M. Mirdita et al., ColabFold: making protein folding accessible to all. Nature Methods 19, 679-682 (2022).
77. Y. Liu et al., CB-Dock: a web server for cavity detection-guided protein-ligand blind docking. Acta Pharmacologica Sinica 41, 138-144 (2020).
78. F. Madeira et al., Search and sequence analysis tools services from EMBL-EBI in 2022. Nucleic acids research, gkac240 (2022).
79. I. Letunic, P. Bork, Interactive Tree Of Life (iTOL) v5: an online tool for phylogenetic tree display and annotation. Nucleic Acids Research 49, W293-W296 (2021).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

1. An engineered bacterial cell or cell lysate for quantitatively monitoring RuBisCO-dependent CO₂ fixation, comprising (a) mutations in the genome that result in bypassing canonical glycolysis pathway and no production or accumulation of 3-phosphoglycerate (3PG) from glycolysis, thereby insulating host metabolism from RuBisCO-produced products, (b) an artificial biosynthetic pathway that produces RuBisCO substrate ribulose 1,5-bisphosphate (RuBP) with reduced toxicity, and (c) a transcriptional biosensor for monitoring glycerate generated from RuBisCO-produced 3PG.

2. The engineered bacterial cell or cell lysate of claim 1, wherein the mutations in the genome comprise mutations that disrupt expression of one or more functional proteins encoded by serA, eda-edd, garDPLRK, glcCDEFGB, gudPXD, allS-allD gene cassette, cdaR, ghrA, ghrB, and ytjC.

3. The engineered bacterial cell or cell lysate of claim 1, wherein the artificial biosynthetic pathway expresses a reduced level of phosphoribulokinase (Prk) relative to Prk expression level in wildtype cell.

4. The engineered bacterial cell or cell lysate of claim 1, wherein the transcriptional biosensor comprises (a) a phosphatase that specifically de-phosphorylate 3PG to generate glycerate, and (b) a CdaR variant that has enhanced specificity for glycerate relative to wildtype CdaR.

5. A method for quantifying RuBisCO-dependent CO₂ fixation by a RuBisCO enzyme, comprising (a) expressing the RuBisCO enzyme in the engineered bacterial cell or cell lysate of claim 1, (b) culturing the cell under conditions suitable for RuBisCO catalyzed CO₂ fixation, and (c) monitoring the amount of RuBisCO-produced 3PG in the cell; thereby quantifying RuBisCO-dependent CO₂ fixation by the RuBisCO enzyme.

6. The method of claim 5, wherein the amount of RuBisCO-produced 3PG is monitored by quantifying glycerate converted from RuBisCO-produced 3PG with the transcriptional biosensor.

7. A method for evolving a RuBisCO enzyme, comprising (a) expressing one or more variants of a reference RuBisCO enzyme in engineered bacterial cell or cell lysate of claim 1 to generate a plurality of cells, wherein each cell expresses one of the RuBisCO variants, (b) monitoring RuBisCO-dependent CO2 fixation in the plurality of cells, and (c) identifying one cell expressing a RuBisCO variant with enhanced CO2 fixation relative to a control cell expressing the reference RuBisCO enzyme; thereby evolving the RuBisCO enzyme.

8. An engineered E. coli cell that bypasses canonical glycolysis pathway and does not produce or accumulate 3-phosphoglycerate (3PG) from glycolysis, comprising mutations that disrupt expression of one or more functional proteins encoded by serA, eda-edd, garDPLRK, glcCDEFGB, gudPXD, allS-allD gene cassette, cdaR, ghrA, ghrB, and ytjC.

9. The engineered E. coli cell of claim 8, further comprising a mutation that disrupts expression of a functional protein encoded by pgk.

10. The engineered E. coli cell of claim 8, further comprising mutations that disrupt expression of functional proteins encoded by one or more genes selected from the group consisting of gpmA, gpmM, gapA, aceBAK, and gph.

11. The engineered E. coli cell of claim 8, wherein the mutations comprise a deletion in each of said genes.

12. The engineered E. coli cell of claim 8, further comprising a point mutation in the homohexamer interface of methylglyoxal synthase (MgsA).

13. The engineered E. coli cell of claim 12, wherein the point mutation comprises a V111A substitution or a S79P substitution, wherein the amino acid numbering is based on E. coli MgsA with Uniprot ID P0A731 (NCBI Reference Sequence NP_415483.2).

14. The engineered E. coli cell of claim 13, further comprising in its genome the mutations set forth in FIG. 1.

15. The engineered E. coli cell of claim 14, further comprising a vector expressing transcriptional repressors LacI and TetR, LuxCDE, and membrane integrity-responsive LacZ cassette.

16. An engineered E. coli cell, comprising an artificial biosynthetic pathway that produces RuBisCO substrate ribulose 1,5-bisphosphate (RuBP) with reduced toxicity.

17. The engineered E. coli cell of claim 16, wherein the artificial biosynthetic pathway expresses a reduced level of phosphoribulokinase (Prk).

18. The engineered E. coli cell of claim 17, wherein the reduced Prk level is expressed with a vector containing low-copy replication origin, a rhamnose-dependent promoter Pᵣₕₐ, and a weakened RBS.

19. The engineered E. coli cell of claim 17, wherein the Prk is a Synechococcus elongatus Prk variant containing R52A or W140A mutation.

20. (canceled)

21. (canceled)

22. (canceled)
